(19) United States
(12) Patent Application Publication
(10) Pub. No.: US 2016/0379505 A1
(43) Pub. Date: Dec. 29, 2016

(54) MENTAL STATE EVENT SIGNATURE USAGE

(71) Applicant: Affectiva, Inc., Waltham, MA (US)

(72) Inventors: Rana el Kaliouby, Milton, MA (US); Evan Kodra, Waltham, MA (US); Daniel McDuff, Cambridge, MA (US); Thomas James Vandal, Dracut, MA (US)

(21) Appl. No.: 15/262,197

(22) Filed: Sep. 12, 2016

Related U.S. Application Data
(63) Continuation-in-part of application No. 14/796,419, filed on Jul. 10, 2015.
Application No. 14/796,419 is a continuation-in-part of application No. 13/153,745, filed on Jun. 6, 2011.
Application No. 14/796,419 is a continuation-in-part of application No. 14/460,915, filed on Aug. 15, 2014.
Application No. 14/460,915 is a continuation-in-part of application No. 13/153,745, filed on Jun. 6, 2011.
(60) Provisional application No. 62/217,872, filed on Sep. 12, 2015.
(60) Provisional application No. 62/222,518, filed on Sep. 23, 2015.
(60) Provisional application No. 62/265,937, filed on Dec. 10, 2015.
(60) Provisional application No. 62/273,896, filed on Dec. 31, 2015.
(60) Provisional application No. 62/273,896, filed on Dec. 31, 2015.
(60) Provisional application No. 62/301,558, filed on Feb. 29, 2016.
(60) Provisional application No. 62/370,421, filed on Aug. 3, 2016.
(60) Provisional application No. 62/023,800, filed on Jul. 11, 2014.
(60) Provisional application No. 62/047,508, filed on Sep. 8, 2014.
(60) Provisional application No. 62/082,579, filed on Nov. 20, 2014.
(60) Provisional application No. 62/128,974, filed on Mar. 5, 2015.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.
(60) Provisional application No. 61/867,007, filed on Aug. 16, 2013.
(60) Provisional application No. 61/924,252, filed on Jan. 7, 2014.
(60) Provisional application No. 61/916,190, filed on Dec. 14, 2013.
(60) Provisional application No. 61/927,481, filed on Jan. 15, 2014.
(60) Provisional application No. 61/953,878, filed on Mar. 16, 2014.
(60) Provisional application No. 61/972,314, filed on Mar. 30, 2014.
(60) Provisional application No. 62/023,800, filed on Jul. 11, 2014.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.

(51) Int. Cl. G09B 5/02; A61B 5/00; G09B 5/12; A61B 5/16

(52) U.S. Cl. CPC G09B 5/02; A61B 5/165; A61B 5/7282; G09B 5/125

(57) ABSTRACT

Mental state event signatures are used to assess how members of a specific social group react to various stimuli such as video advertisements. The likelihood that a video will go viral is computed based on mental state event signatures. Automated facial expression analysis is utilized to determine an emotional response curve for viewers of a video. The emotional response curve is used to derive a virality probability index for the video. The virality probability index is an indicator of the propensity to go viral for a given video. The emotional response curves are processed according to various demographic criteria in order to account for cultural differences amongst various demographic groups and geographic regions.

### RELATED APPLICATIONS

This application claims the benefit of U.S. provisional patent applications “Mental State Event Signature Usage” Ser. No. 62/217,872, filed Sep. 12, 2015, “Image Analysis In Support of Robotic Manipulation” Ser. No. 62/222,518, filed Sep. 23, 2015, “Analysis of Image Content with Associated Manipulation of Expression Presentation” Ser. No. 62/265,937, filed Dec. 12, 2015, “Image Analysis Using Sub-Sectional Component Evaluation To Augment Classifier Usage” Ser. No. 62/273,896, filed Dec. 31, 2015, “Analytics for Live Streaming Based on Image Analysis within a Shared Digital Environment” Ser. No. 62/301,558, filed Feb. 29, 2016, and “Deep Convolutional Neural Network Analysis of Images for Mental States” Ser. No. 62/370,421, filed Aug. 3, 2016.

This application is also a continuation-in-part of U.S. patent application “Mental State Event Definition Generation” Ser. No. 14/796,419, filed Jul. 10, 2015 which claims the benefit of U.S. provisional patent applications “Mental State Event Definition Generation” Ser. No. 62/023,800, filed Jul. 11, 2014, “Facial Tracking with Classifiers” Ser. No. 62/047,508, filed Sep. 8, 2014, “Semiconductor Based Mental State Analysis” Ser. No. 62/082,579, filed Nov. 20, 2014, and “Viewership Analysis Based On Facial Evaluation” Ser. No. 62/128,974, filed Mar. 5, 2015. The patent application “Mental State Event Definition Generation” Ser. No. 14/796,419, filed Jul. 10, 2015 is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. The patent application “Mental State Event Definition Generation” Ser. No. 14/796,419, filed Jul. 10, 2015 is also a continuation-in-part of U.S. patent application “Mental State Analysis Using an Application Programming Interface” Ser. No. 14/460,915, Aug. 15, 2014, which claims the benefit of U.S. provisional patent applications “Application Programming Interface for Mental State Analysis” Ser. No. 61/867,007, filed Aug. 16, 2013, “Mental State Analysis Using an Application Programming Interface” Ser. No. 61/924,252, filed Jan. 7, 2014, “Heart Rate Variability Evaluation for Mental State Analysis” Ser. No. 61/916,190, filed Dec. 14, 2013, “Mental State Analysis for Norm Generation” Ser. No. 61/927,481, filed Jan. 15, 2014, “Expression Analysis in Response to Mental State Express Request” Ser. No. 61/953,878, filed Mar. 16, 2014, “Background Analysis of Mental State Expressions” Ser. No. 61/972,314, filed Mar. 30, 2014, and “Mental State Event Definition Generation” Ser. No. 62/023,800, filed Jul. 11, 2014. The patent application “Mental State Event Definition Generation” Ser. No. 14/796,419, filed Jul. 10, 2015 is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. The foregoing applications are each hereby incorporated by reference in their entirety.

### FIELD OF ART

This application relates generally to mental state analysis and more particularly to mental state event signature usage.

### BACKGROUND

Psychology plays a large role in many facets of today's society. Activities such as advertising, product promotion, sales, marketing, and recruiting can all be correlated with various psychological principles. That is, the mental state of a person can vary upon being subject to advertising or other persuasive campaigns and can represent a key factor in determining the success of such endeavors. On a more basic level, individuals have mental states that vary in response to various situations in life. While an individual's mental state is important to general well-being and impacts his or her decision making, multiple individuals' mental states resulting from a common event can carry a collective importance that, in certain situations, is even more important than the individual's mental state taken alone. Mental states include a wide range of emotions and experiences from happiness to sadness, from contentedness to worry, from excitation to calm, and many others. Despite the importance of mental states in daily life, the mental state of even a single individual might not always be apparent, even to the individual in question. Before even discussing the process of determining the mental states of a collective group, it must be noted that the ability and means by which even one person perceives his or her emotional state can be quite difficult to summarize. Though an individual can often perceive his or her own emotional state quickly, instinctively and with a minimum of conscious effort, the individual might encounter difficulty when attempting to summarize or communicate his or her mental state to others. The problem of understanding and communicating mental states becomes even more difficult when the mental states of multiple individuals are considered.

Gaining insight into the mental states of multiple individuals represents an important tool for understanding events. For example, advertisers seek to understand the resultant mental states of viewers of their advertisements in order to gauge the efficacy of those advertisements. However, it is very difficult to properly interpret mental states when the individuals under consideration might themselves be unable to accurately communicate their mental states.

Adding to the difficulty is the fact that multiple individuals can have similar or very different mental states when taking part in the same shared activity. For example, the mental state of two friends can be very different after a certain team wins an important sporting event. Clearly, if one friend is a fan of the winning team, and the other friend is a fan of the losing team, widely varying mental states can be expected. However, the problem of defining the mental states of more than one individual to stimuli more complex than a sports team winning or losing can prove a much more difficult exercise in understanding mental states.

Ascertaining and identifying multiple individuals' mental states in response to a common event can provide powerful insight into both the impact of the event and the individuals' mutual interaction and communal response to the event. Thus, analysis of mental states in response to certain events can provide important information with both social and financial implications.

### SUMMARY

Disclosed embodiments provide a computer-implemented method for analysis of event signatures. The event signatures can be generated by automated methods, manual methods, or a combination of automated and manual methods. Regardless of how the event signatures are generated, they can be used for a variety of purposes, such as gauging the efficacy of advertisements or the likelihood that a video will go viral. As advertising campaigns can cost millions of dollars, methods and systems for assessing the efficacy of such advertisements, videos, and other promotional material provide valuable feedback for the stakeholders in those advertising campaigns. Embodiments have uses beyond analysis of advertising. For example, public service announcements, safety instructions, movies, television shows, live theater, music performances, poetry performances, political speeches, and other forms of media and artistic expression can be evaluated using disclosed embodiments. Furthermore, embodiments can have applications in psychology, social skills training, and mental health. A computer-implemented method for analysis is disclosed comprising: obtaining a plurality of mental state event temporal signatures; collecting mental state data from an individual; comparing the plurality of mental state event temporal signatures against the mental state data; and identifying a mental state event type, based on the plurality of mental state event temporal signatures. The method can include using the mental state event type, which was identified, to perform an evaluation of the individual against other people within a social group. In embodiments, the method can include determining a significant difference for the mental state data for the individual versus the social group. In some embodiments, the method includes comparing the individual against a norm for the social group.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for event signature usage.

FIG. 2 is a flow diagram for event signature matching.

FIG. 3 is a diagram showing cameras obtaining images of a person.

FIG. 4 shows an example of image collection from multiple mobile devices.

FIG. 5 shows an example of clustering by parameter.

FIG. 6 shows an example plot for smile peak and duration.

FIG. 7 is an example plot showing peak rise time of smiles.

FIG. 8 is an example of a stacked bar chart showing smile event clusters by region.

FIG. 9 is an example of a stacked area chart showing smile event occurrences.

FIG. 10 shows example of facial data collection including landmarks.

FIG. 11 is a flow diagram for detecting facial expressions.

FIG. 12 is a flow diagram for large-scale clustering of facial events.

FIG. 13 shows an example of unsupervised clustering of features and characterizations of cluster profiles.

FIG. 14A shows an example of tags embedded in a webpage.

FIG. 14B shows an example of invoking tags for the collection of images.

FIG. 15 shows an example of live-streaming of social video.

FIG. 16 is a system diagram for mental state event signature usage.

### DETAILED DESCRIPTION

Disclosed embodiments use event signatures to determine a collective response to an event. The event can be a promotional event, including, but not limited to, an advertisement for a product, a recruitment advertisement (e.g. soliciting the individual to apply for a job, join a club or group, etc.), a request for donations, or a public service announcement, to name a few. In addition, disclosed embodiments utilize event signatures associated with demographic information, such as country or region of residence. Due to cultural differences, people from one country or region might respond differently than people from other regions. Therefore, an advertisement that is effective in one market might not be as effective in other regions/markets, due in part to cultural differences. Understanding demographic factors is important for generating effective promotional/persuasive content in today's globally connected world.

Large datasets are useful for generating meaningful data such as event signatures, which show the response of a group of people to a given event. The event signatures can be based on facial expressions of a sampling of people as they experience an event. With automated facial expression identification, it is possible to collect and analyze data on a large scale. While collection of large amounts of data is helpful for understanding the collective feelings of a group, it is in the usage of such data that new levels of analysis of mental states based on demographics can be achieved. Advances in the ways in which such data can be used have implications in advertising, education, political discourse, treatment and diagnosis of mental illness, and a variety of other important applications.

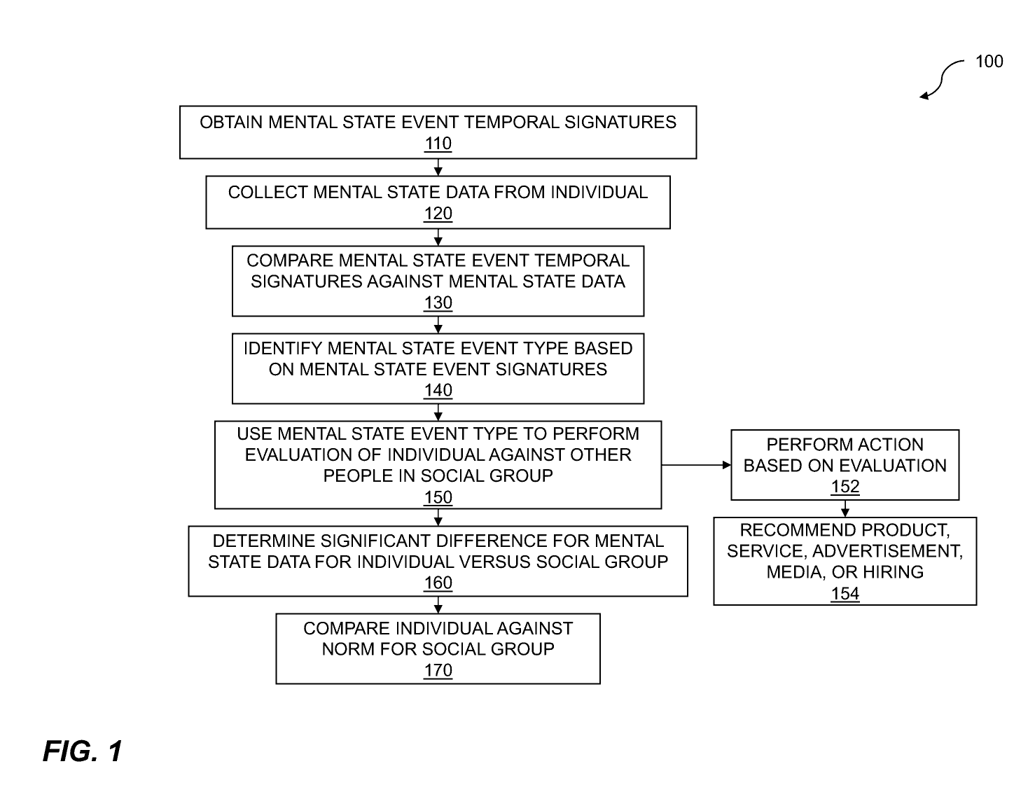

FIG. 1 is a flow diagram for event signature usage. The flow 100 describes a computer-implemented method for analysis. The flow 100 includes obtaining a plurality of mental state event temporal signatures 110. The mental state event temporal signatures indicate a collective mental state of a group of people over a period of time as the group experiences an event. For example, the group of people might be asked to view a video. During the course of the video, the mental state of the group of people can be assessed, and a signature can be obtained. For example, the signature can indicate a smile intensity that can correlate to a particular point in the video that the subjects are viewing.

The flow 100 includes collecting mental state data from an individual 120. For example, an individual can be asked to watch a video, and mental state data can then be collected from the individual as the video is being viewed. The collecting can be performed in an automated manner using facial recognition systems, image classifiers, and other suitable techniques.

The flow 100 includes comparing the plurality of mental state event temporal signatures against the mental state data 130. The flow 100 includes identifying a mental state event type, based on the plurality of mental state event signatures 140. The event type can be a detection of a particular emotional state, such as happiness, fear, or surprise, to name a few.

The flow 100 includes using the mental state event type which was identified to perform an evaluation of the individual against other people within a social group 150. The evaluation can be used to determine if the individual is behaving similarly to members of the group from which the mental state event temporal signatures were derived. Various pieces of information can be obtained from the evaluation technique. For example, if a person was born and raised in a first geographical region (Region A), and later moved to a second geographical region (Region B), disclosed embodiments can determine if the person reacts more like someone from Region A or from Region B. Additionally, mental state information can be collected from people who moved from Region A to Region B at various ages to determine a transition age defined as the age beyond which the subject is more likely to exhibit characteristics of Region A. Region A and Region B can be international regions, such as, for example, India and the United States respectively. Alternatively, Region A and Region B could both be within the United States. For example, Region A could be New England, and Region B could be the Mid-Atlantic States. Referring again to the international example of Region A as India and Region B as the United States, a series of experiments evaluating the mental state event type of individuals who moved from India to the United States at various ages might determine a transition age of 13 years. This determination would infer that people who move from India to the United States before the age of 13 are inclined to react similarly to people from the United States. Conversely, it can also be inferred that if a person moves from India to the United States after age 13, they are more likely to react similarly to people from India. Different regions and social groups can have different transition ages. Social groups can be based on demographics, income, job responsibilities, ethnicity, buying behavior, or career objectives.

The flow 100 can continue with performing an action based on an evaluation 152. The flow can further include recommending a product, service, advertisement, media, or hiring 154. Thus, in embodiments, the action includes recommending a product, recommending a service, providing an advertisement, recommending media, or recommending for hiring. For example, in embodiments, a user can be asked to view a video, taste food, listen to audio, or undergo another experience. The collected mental state data from the individual, upon being compared against other people in a social group, can be used as criteria in performing an action. The action can include a screening effort of videos in preparation for human analysis. Thus, embodiments provide an automated prescreening function for videos that include facial expressions for analysis. A subset of those videos can be flagged for human analysis by the automated system. The criteria for flagging videos can include, but is not limited to, random selection, selecting videos based on how many mental state transitions are detected within the video, and/or videos containing mental states that the automated system cannot recognize. Thus, in some embodiments, the event signatures are derived by a combination of automated (computer-implemented) and manual (human-based) methods.

The flow comprises determining a significant difference for the mental state data for an individual versus a social group 160. The flow continues with comparing the mental state data for the individual against a norm for the social group 170. Naturally, an individual's mental state might not always align with the norm for the social group to which they belong. Thus in some cases, a user's reaction deviates from what is expected from their social group. This deviation can be used as an input for a targeted advertisement system. For example, consider a social group made up of males aged 18 to 25. By default, a targeted advertisement system might present a default advertisement to all people within this social group. However, based on the comparing of an individual against a norm for a social group, a particular user can be presented with an alternate advertisement. Thus, in embodiments, performing an action is based on the evaluation of the individual against the other people. The targeted advertising system can be a web-based system for delivering advertisements to a computer browser, a mobile browser, or can deliver individualized advertisements on some other platform, such as internet radio, satellite radio, cable television, and/or satellite television.

A subtle response can be normative for the social group and a more expressive response can be provided by the individual where detection of the more expressive response can be used in the identifying of the mental state event type. That is, suppose that two group event signatures are obtained from social groups of people born/raised in region A and region B respectively. It might be found that people from region A tend to smile often and with considerable intensity, while people from region B tend to have a muted reaction and only a very slight smile even when quite happy. The difference in reaction can be due to cultural differences between region A and region B. However, if a person from region B has a smile similar to the intensity that is normative for region A, then the expression can be given additional weight. Thus, a numerical multiplier can be used for generating intensity data for expressions for people belonging to social group B. The implementation of a multiplier allows cultural differences to be factored into facial expression analysis by compensating for the fact that the more expressive response from a member of the social group of region B might reflect more emotional intensity than a similar response from a member of the social group of region A. Thus a first response level can be normative for the social group and a second response level can be provided by the individual where detection of the second response level is used in the identifying of the mental state event type. The first response level can be a subtle response relative to second response level which is a more expressive response. The providing of a type of feedback action 152 can be based on a normative score based on the mental state event type. Thus, a score based on a particular intensity and/or duration of facial expression, if exceeding a predetermined threshold, can trigger an action. The action can include computing a virality probability index for a video viewed by the individual while the mental state data is being collected. In current Internet culture, viral videos (videos that quickly spread across the Web) can have considerable economic value. Therefore, it is desirable to have embodiments that can serve to predict the likelihood of a video going viral. Assessing the mental state of viewers of such a video, among other things, can be used as a criterion for computing a virality probability index for a given video. Videos with a virality probability index above a predetermined threshold can be categorized as having an increased probability to go viral. For advertisements, the virality probability index can have important financial and economic implications.

The steps described in the flow 100 serve to outline methods and systems for using mental state event temporal signatures to analyze a group response to an event and to calculate, taking into account demographic and cultural factors, meaningful individual deviations from the group response and to take various actions based on deviations when desired. Various steps in the flow 100 may be changed in order, repeated, omitted, or the like without departing from the disclosed concepts. Various embodiments of the flow 100 may be included in a computer program product embodied in a non-transitory computer readable medium that includes code executable by one or more processors.

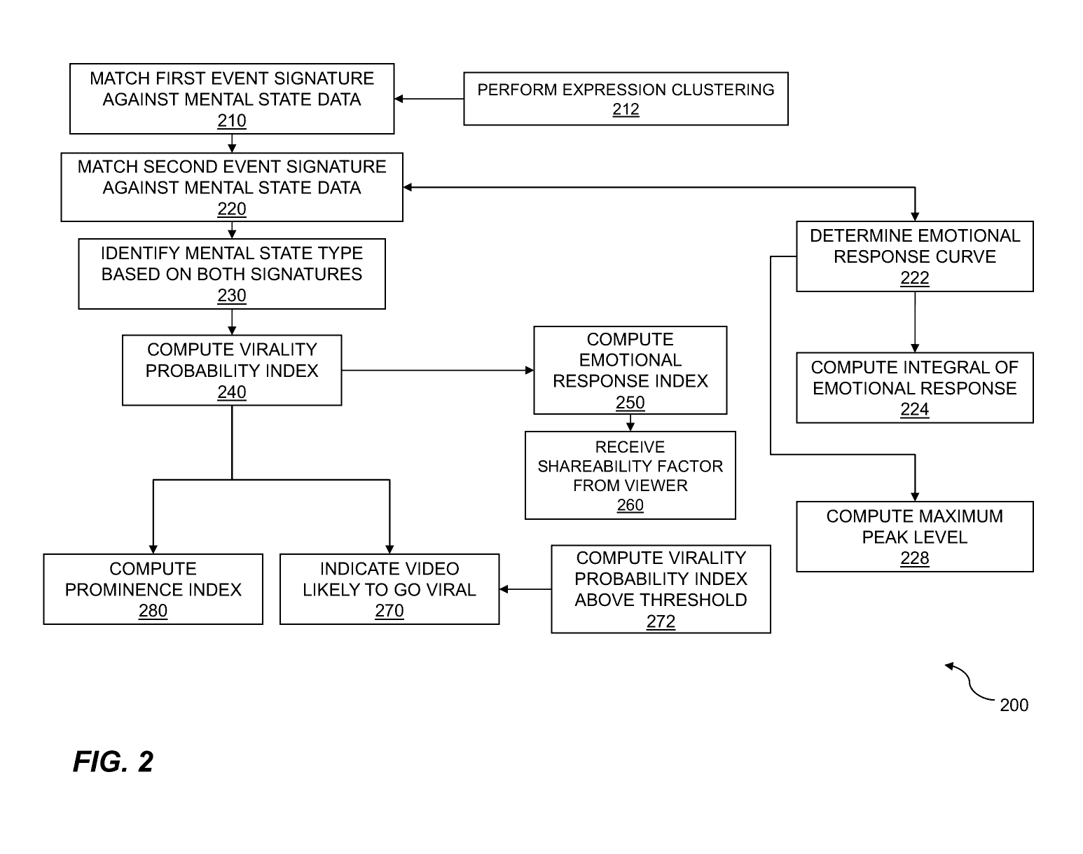

FIG. 2 is a flow diagram for event signature matching. The flow 200 describes matching two distinct event signatures against mental state data for the purpose of predicting and computing audience response or interest in a presentation. The flow 200 includes matching a first event signature against mental state data 210. Thus, embodiments include matching a first event signature, from the plurality of mental state event signatures, against the mental state data that was obtained. The first event signature can be used by an SDK (software development kit). Thus, embodiments provide a library and a set of interfaces (APIs) for accessing the event signature. The provision of libraries and interfaces allows embodiments to be integrated with systems such as targeted advertising systems, rating services, and other applications where the mental state data is relevant. The first event signature can be used on a mobile device. The device can include, but is not limited to, a smart phone, tablet, or wearable computer, or another portable device. In embodiments, the first event signature is based on an image classifier and includes a peak intensity and a duration for an expression. In embodiments, the first event signature is obtained by performing expression clustering, and, as a result, the flow 200 can include performing expression clustering 212. Expression clustering can be performed for a variety of purposes including mental state analysis. The expression clustering can include a variety of facial expressions and can be for smiles, smirks, brow furrows, squints, lowered eyebrows, raised eyebrows, or attention. The expression clustering can be based on action units (AUs), with any appropriate AUs able to be considered for the expression clustering such as inner brow raiser, outer brow raiser, brow lowerer, upper lid raiser, cheek raiser, lid tightener, lips toward each other, nose wrinkle, upper lid raiser, nasolabial deepener, lip corner puller, sharp lip puller, dimpler, lip corner depressor, lower lip depressor, chin raiser, lip pucker, tongue show, lip stretcher, neck tightener, lip funneler, lip tightener, lips part, jaw drop, mouth stretch, lip suck, jaw thrust, jaw sideways, jaw clencher, [lip] bite, [cheek] blow, cheek puff, cheek suck, tongue bulge, lip wipe, nostril dilator, nostril compressor, glabella lowerer, inner eyebrow lowerer, eyes closed, eyebrow gatherer, blink, wink, head turn left, head turn right, head up, head down, head tilt left, head tilt right, head forward, head thrust forward, head back, head shake up and down, head shake side to side, head upward and to the side, eyes turn left, eyes left, eyes turn right, eyes right, eyes up, eyes down, walleye, cross-eye, upward rolling of eyes, clockwise upward rolling of eyes, counter-clockwise upward rolling of eyes, eyes positioned to look at other person, head and/or eyes look at other person, sniff, speech, swallow, chewing, shoulder shrug, head shake back and forth, head nod up and down, flash, partial flash, shiver/tremble, or fast up-down look. The classifiers can be implemented in such a way that the expression clustering can be based on the analyzing of the videos using the classifiers, but the expression clustering can also be based on self-reporting by the people from whom the videos were obtained, including self-reporting performed by an online survey, a survey app, a web form, a paper form, and so on. The self-reporting can take place immediately following the obtaining of the video of the person, or at another appropriate time, for example.

The flow 200 can further include matching a second event signature against mental state data 220. Thus, embodiments comprise matching a second event signature, from the plurality of mental state event signatures, against the mental state data that was obtained and identifying the mental state event type based on both the first event signature and second event signature. The event signature can be used to detect one or more of sadness, stress, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, curiosity, humor, poignancy, or mirth. The first event signature can correspond to a first time period and the second event signature can correspond to a second time period. The first and second event signatures can be used to track changes in mental states. For example, while watching a video, a user might first provide facial expressions indicative of confusion, and then at another point in the video, when the confusion is resolved, the user might provide facial expressions indicative of a second mental state (e.g. happiness). The use of multiple event signatures allows tracking changes in mental states during the course of an experience.

The first event signature can further include a rise rate to the peak intensity, a fall rate from the peak intensity, a trough value for intensity, a delta between the trough value for the intensity and the peak intensity, a time delta between the trough value and the peak intensity, a trough value for intensity after peak value, a delta between the peak intensity and the trough value for the intensity after the peak value, a time delta between the peak intensity and the trough value, a time delta between a trough value before the peak intensity and the trough value after the peak value, a beginning of onset and an end of onset timing, a beginning of offset and an end of offset timing, or a sustained period timing.

The flow 200 can continue with determining an emotional response curve 222. The emotional response curve can represent emotional intensity as a function of time. The emotional intensity can be based on a particular facial expression, such as a smile. In such embodiments, a large smile correlates to a high intensity, whereas a flat expression corresponds to a low intensity. The emotional response curve 222 can be analyzed in multiple ways. The flow can continue with computing an integral of the emotional response curve 224. The computed integral considers the area under the emotional response curve, and thus is a function of the duration of the expression. Thus, an intense smile for several seconds results in a greater integral value than a smile of similar intensity for a brief time (e.g. 500 milliseconds). Therefore, in embodiments, the computing of the emotional response index comprises determining an emotional response curve as a function of time for the video, and computing an integral of the emotional response curve.

The flow 200 can also include computing a maximum peak level 228. The maximum peak level takes into account the amplitude of the emotional response curve but is not a function of expression duration. Maximum peak level, an integral between two local minima, or a combination of peak and integral can be used in evaluating the emotional response curve. Thus, the computing of the emotional response index can comprise determining an emotional response curve 222 as a function of time for the video and computing an integral of the emotional response curve 224. Additionally, the computing of the emotional response index can comprise determining an emotional response curve 224 as a function of time for the video and computing a maximum peak level 228 for the emotional response curve. The first event signature can be based on an image classifier and can include a peak intensity and a duration for an expression.

The flow continues with identifying a mental state type based on both signatures 230. The mental state type can be a compound mental state type that includes at least one mental state transition. Example compound mental state types include, but are not limited to, happy-angry (i.e. transitioning from a happy mental state to an angry mental state), angry-happy, confused-angry, sad-happy, and so on. Additionally, more than two signatures can be used to analyze larger compound mental states, such as confused-angry-happy, concerned-confused-happy, happy-angry-happy, etc. Many compound mental state types can be identified with embodiments.

The flow 200 continues with computing a virality probability index 240. The virality probability index is an indicator of how likely it is that a video (or other piece of media such as a song or picture) is likely to go viral. In embodiments, the virality probability index V is computed as:

V=K₁(P)+K₂(I)

where P is a peak level of an emotional response curve, I is an integral of an emotional response curve, and K₁ and K₂ are constants. The peak level can be a nominal level that ranges from zero (no intensity) to a maximum intensity of 10. A predetermined virality index threshold can be established. If a virality index of a particular video exceeds the established threshold, the video can be considered likely to go viral. In the particular example given below, the virality index threshold is 100, the constant K₁=5, and the constant K₂=3. This results in the following, based on the sample data shown below:

| Video | Peak | Integral | Virality Probability Index |
|---|---|---|---|
| 1 | 8 | 21 | 103 |
| 2 | 5 | 11 | 58 |
| 3 | 2 | 5 | 25 |
| 4 | 7 | 28 | 119 |

The computing of the virality probability index 240 can further comprise computing an emotional response index 250 for the video. In the previous example, the portion of the expression K₁(P)+K₂(I) represents an emotional response index. In some embodiments, the virality probability index is based on other factors in addition to the emotional response index. The computing of the virality probability index can further comprise receiving a shareability factor from a viewer 260 of the video. The received shareability factor can be based on self-reporting by the individual. For example, after watching a video, a user might be asked how likely they are to share the video, on a scale from 1 (would not share) to 10 (would definitely share). The shareability factor can be included as part of the virality probability index formula. In one embodiment, the virality probability index V is computed as:

V=K₁(P)+K₂(I)+K₃(S)

where K₃ is a constant and S is the shareability factor.

The computing of the virality probability index 240 can further comprise indicating a video that is likely to go viral 270 in response to computing a virality probability index above a predetermined threshold 272. Thus in the example previously disclosed, video 1 and video 4 both have a virality probability index above the predetermined threshold of 100, and thus are deemed likely to go viral, whereas videos 2 and 3 both have a virality probability index below 100, and thus are deemed not likely to go viral.

The computing of the virality probability index 240 can further comprise computing a prominence index 280 for subjects shown in the video. In embodiments, the prominence index is a measure of the subjects within the video. The subjects can include people, places and things. The prominence index can be derived by a combination of how famous a subject is, and how long that subject appears in the video. For example, in the case of a famous person, a fame factor F indicative of their level of fame/relevance can be ranked on a scale from 1 (not famous/relevant) to 10 (very famous/relevant). In embodiments, the computing of the virality probability index further comprises computing a prominence index for people seen in the video. The prominence factor can be further derived from a duration percentage D, reflecting the percentage of time that the famous person appears in the video. Thus, in an embodiment, the prominence index R is defined as:

R=K₄(F)+K₅(D)

where K₄ and K₅ are constants, F is the fame factor, and D is the duration percentage. The resultant equation can then be incorporated into a virality probability index as follows:

V=K₁(P)+K₂(I)+K₃(S)+K₆(R)

where K₁, K₂, K₃, and K₆ are constants, P is a peak level of an emotional response curve, I is an integral of an emotional response curve, S is a shareability factor (how likely the user is to share the video), and R is the prominence index (how famous/relevant the subjects of the video are, and for how long the subjects appear in the video).

Emotion analysis results can be communicated in various ways. Graphical or visual representations can be provided. A representative icon can be provided such as a character, a pictograph, an emoticon, and so on. The representative icon can include an emoji. One or more emoji can be used to represent a mental state, a mood, etc. of an individual; to represent food, a geographic location, weather, and so on. The emoji can include a static image. The static image can be a predefined size such as a number of pixels, for example. The emoji can include an animated image. The emoji can be based, for example, on a GIF or another animation standard. The emoji can include a cartoon representation. The cartoon representation can be various cartoon types, formats, etc. that can be appropriate to representing an emoji.

The methods and systems described in diagram 200 thus provide for the calculating of a virality index and probability index based on combinational data from two different mental state signatures. Various steps in the flow 200 may be changed in order, repeated, omitted, or the like without departing from the disclosed concepts. Various embodiments of the flow 200 may be included in a computer program product embodied in a non-transitory computer readable medium that includes code executable by one or more processors.

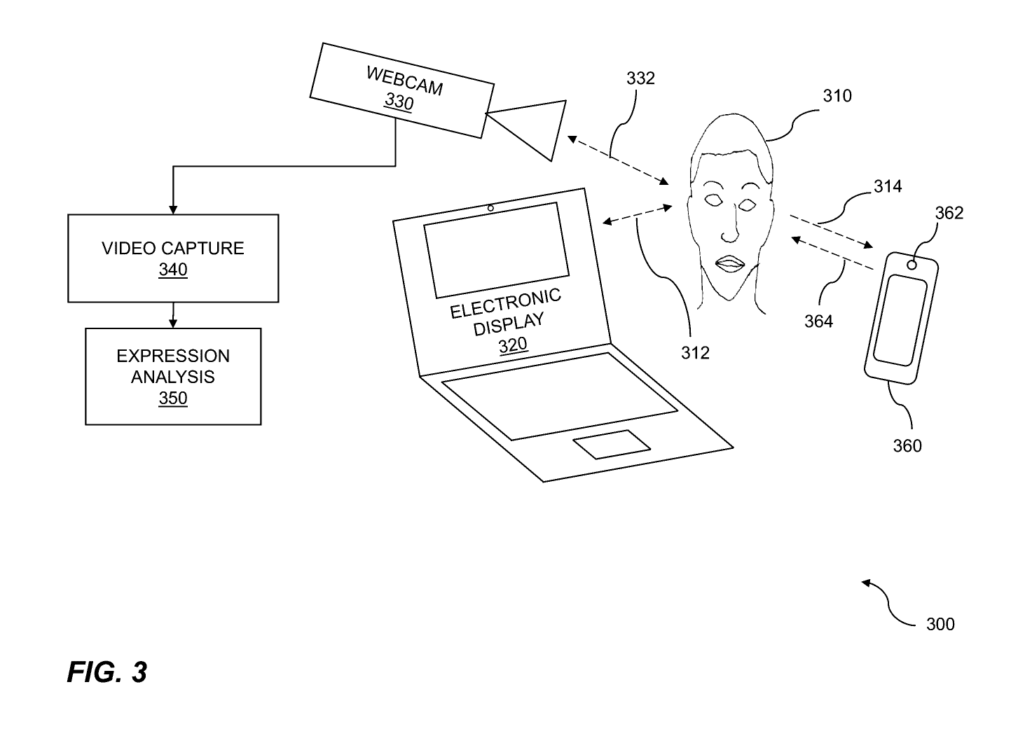

FIG. 3 is a diagram showing cameras obtaining images of a person. These images can be used in mental state event signature analysis. The image capture can be performed for the purpose of obtaining mental state data from the person. The example 300 shows a person 310 viewing an event on one or more electronic displays. In practice, any number of displays can be shown to the person 310. An event can be a media presentation, where the media presentation can be viewed on an electronic display. The media presentation can be an advertisement, a political campaign announcement, a TV show, a movie, a video clip, or any other type of media presentation. In the example 300, the person 310 has a line of sight 312 to an electronic display 320. Similarly, the person 310 has a line of sight 314 to a display of a mobile device 360. While one person has been shown, in practical use, embodiments of the present invention can analyze groups comprising tens, hundreds, or thousands of people or more. In embodiments including groups of people, each person has a line of sight 312 to the event or media presentation rendered on an electronic display 320 and/or each person has a line of sight 314 to the event or media presentation rendered on an electronic display of a mobile device 360. The plurality of captured videos can be of people who are viewing substantially identical media presentations or events, or conversely, the videos can capture people viewing different events or media presentations.

The display 320 can comprise a television monitor, a projector, a computer monitor (including a laptop screen, a tablet screen, a net book screen, and the like), a projection apparatus, and the like. The display of the device 360 can be a cell phone display, a smartphone display, a mobile device display, a tablet display, or another electronic display. A camera can be used to capture images and video of the person 310. In the example 300 shown, a webcam 330 has a line of sight 332 to the person 310. In one embodiment, the webcam 330 is a networked digital camera that can take still and/or moving images of the face and possibly the body of the person 310. The webcam 330 can be used to capture one or more of the facial data and the physiological data. Additionally, the example 300 shows a camera 362 on a mobile device 360 with a line of sight 364 to the person 310. As with the webcam, the camera 362 can be used to capture one or more of the facial data and the physiological data of the person 310.

The webcam 330 can be used to capture data from the person 310. The webcam 330 can be any camera including a camera on a computer 320 (such as a laptop, a net book, a tablet, or the like), a video camera, a still camera, a 3-D camera, a thermal imager, a CCD device, a three-dimensional camera, a light field camera, multiple webcams used to show different views of the viewers, or any other type of image capture apparatus that allows captured image data to be used in an electronic system. In addition, the webcam can be a cell phone camera, a mobile device camera (including, but not limited to, a forward-facing camera), and so on. The webcam 330 can capture a video or a plurality of videos of the person or persons viewing the event or situation. The plurality of videos can be captured of people who are viewing substantially identical situations, such as viewing media presentations or events. The videos can be captured by a single camera, an array of cameras, randomly placed cameras, a mix of different types of cameras, and so on. As mentioned above, media presentations can comprise an advertisement, a political campaign announcement, a TV show, a movie, a video clip, or any other type of media presentation. The media can be oriented toward an emotion. For example, the media can include comedic material to evoke happiness, tragic material to evoke sorrow, and so on.

The facial data from the webcam 330 is received by a video capture module 340 which can decompress the video into a raw format from a compressed format such as H.264, MPEG-2, or the like. Received facial data can be received in the form of a plurality of videos, with the possibility of the plurality of videos coming from a plurality of devices. The plurality of videos can be of one person and of a plurality of people who are viewing substantially identical situations or substantially different situations. The substantially identical situations can include viewing media, listening to audio-only media, and/or viewing still photographs. The facial data can include information on action units, head gestures, eye movements, muscle movements, expressions, smiles, and the like.

The raw video data can then be processed for expression analysis 350. The processing can include analysis of expression data, action units, gestures, mental states, and so on. Facial data as contained in the raw video data can include information on one or more of action units, head gestures, smiles, brow furrows, squints, lowered eyebrows, raised eyebrows, attention, and the like. The action units can be used to identify smiles, frowns, and other facial indicators of expressions. Gestures can also be identified, and can include a head tilt to the side, a forward lean, a smile, a frown, as well as many other gestures. Other types of data including physiological data can be obtained, where the physiological data is obtained through the webcam 330 without contacting the person or persons. Respiration, heart rate, heart rate variability, perspiration, temperature, and other physiological indicators of mental state can be determined by analyzing the images and video data. Using the methods described above, mental state data of various types and for various uses can be collected from an individual or a group of individuals.

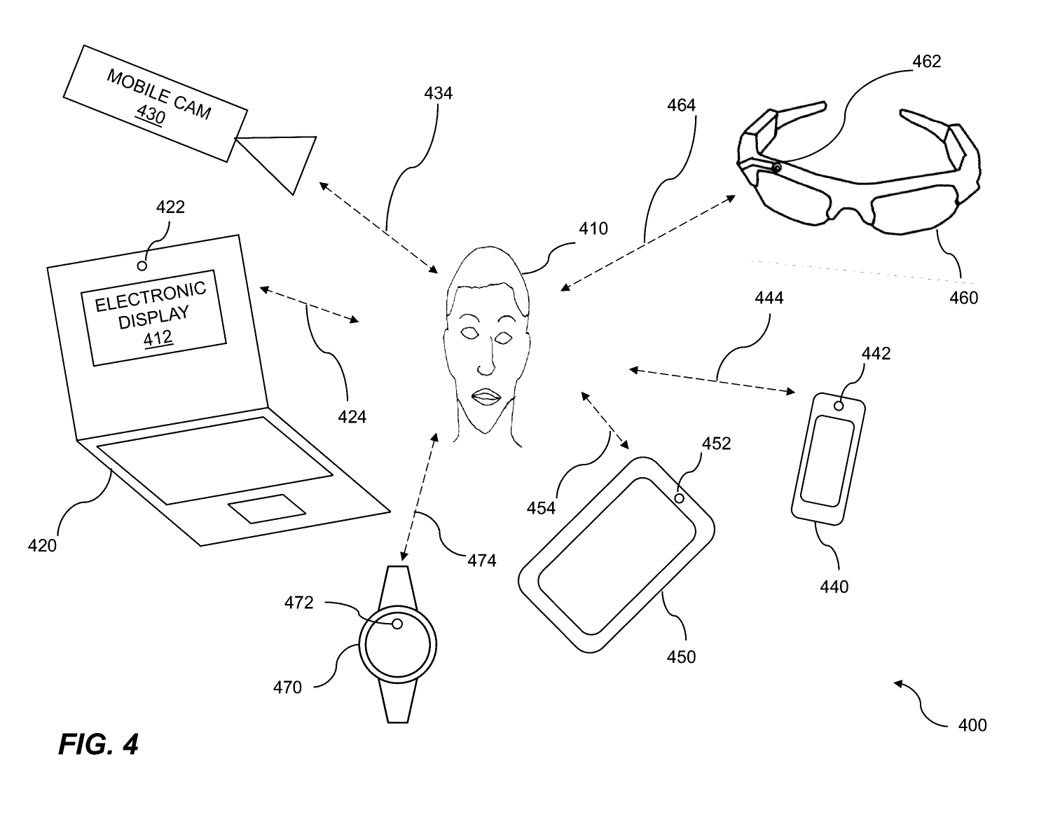

FIG. 4 is a diagram 400 showing example image collection from multiple mobile devices. Images from multiple sources can be used in mental state event signature analysis. The multiple mobile devices can be used to collect video data on a person. While one person is shown, in practice, video data can be collected on any number of people. A user 410 can be observed as she or he is performing a task, experiencing an event, viewing a media presentation, and so on. The user 410 can be shown one or more media presentations, for example, or another form of displayed media. The one or more media presentations can be shown to a plurality of people instead of an individual user. The media presentations can be displayed on an electronic display 412. The data collected on the user 410 or on a plurality of users can take the form of one or more videos. The plurality of videos can be of people who are experiencing different situations. Some example situations can include the user or plurality of users being exposed to TV programs, movies, video clips, and other similar media. The situations could also include exposure to media such as advertisements, political messages, news programs, and so on. As noted before, video data can be collected on one or more users in substantially identical or different situations who are viewing either a single media presentation or a plurality of presentations. The data collected on the user 410 can be analyzed and viewed for a variety of purposes, including expression analysis. The electronic display 412 can be on a laptop computer 420 as shown, a tablet computer 450, a cell phone 440, a television, a mobile monitor, or any other type of electronic device. In a certain embodiment, expression data is collected on a mobile device such as a cell phone 440, a tablet computer 450, a laptop computer 420, or a watch 470. Thus, the multiple sources can include at least one mobile device such as a phone 440 or a tablet 450, or a wearable device such as a watch 470 or glasses 460. A mobile device can include a forward-facing camera and/or a rear-facing camera that can be used to collect expression data. Sources of expression data can include a webcam 422, a phone camera 442, a tablet camera 452, a wearable camera 462, and a mobile camera 430. A wearable camera can comprise various camera devices such as the watch camera 472.

As the user 410 is monitored, the user 410 might move due to the nature of the task, boredom, discomfort, distractions, or for other reasons. As the user moves, the camera with a view of the user's face can change. Thus, as an example, if the user 410 is looking in a first direction, the line of sight 424 from the webcam 422 is able to observe the individual's face, but if the user is looking in a second direction, the line of sight 434 from the mobile camera 430 is able to observe the individual's face. Further, in other embodiments, if the user is looking in a third direction, the line of sight 444 from the phone camera 442 is able to observe the individual's face, and if the user is looking in a fourth direction, the line of sight 454 from the tablet camera 452 is able to observe the individual's face. If the user is looking in a fifth direction, the line of sight 464 from the wearable camera 462, which can be a device such as the glasses 460 shown and can be worn by another user or an observer, is able to observe the individual's face. If the user is looking in a sixth direction, the line of sight 474 from the wearable watch-type device 470 with a camera 472 is able to observe the individual's face. In other embodiments, the wearable device is another device, such as an earpiece with a camera, a helmet or hat with a camera, a clip-on camera attached to clothing, or any other type of wearable device with a camera or other sensor for collecting expression data. The user 410 can also employ a wearable device including a camera for gathering contextual information and/or collecting expression data on other users. Because the user 410 can move her or his head, the facial data can be collected intermittently when the individual is looking in a direction of a camera. In some cases, multiple people are included in the view from one or more cameras, and some embodiments include filtering out faces of one or more other people to determine whether the user 410 is looking toward a camera. All or some of the expression data can be continuously or sporadically available from these various devices and other devices.

The captured video data can include facial expressions and can be analyzed on a computing device such as the video capture device or on another separate device. The analysis of the video data can include the use of a classifier. For example, the video data can be captured using one of the mobile devices discussed above and sent to a server or another computing device for analysis. However, the captured video data including expressions can also be analyzed on the device which performed the capturing. For example, the analysis can be performed on a mobile device where the videos were obtained with the mobile device and wherein the mobile device includes one or more of a laptop computer, a tablet, a PDA, a smartphone, a wearable device, and so on. In another embodiment, the analyzing can comprise using a classifier on a server or other computing device other than the capturing device.

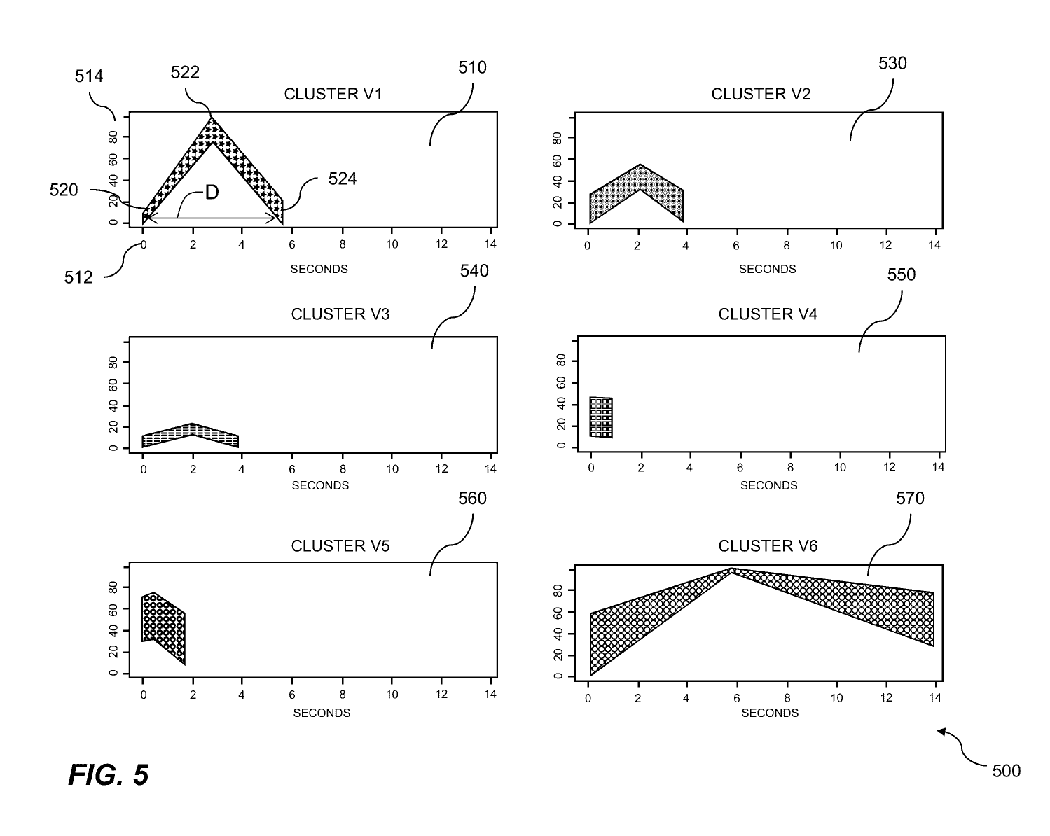

FIG. 5 shows example clustering by parameter. In the example graphs 500 shown, smile intensities are given to illustrate changes and therefore possible components of expression signatures. A component can be a peak intensity value, a difference between a trough and a peak value, a rate of expression change rising towards the peak or descending from the peak, a duration of intensity, and so on. In embodiments, the following signature attributes are tracked: Event Height (maximum value), Event Length (duration between onset and offset), Event Rise (increase from onset to peak), Event Decay (decrease from peak to next offset), Rise Speed (gradient of event rise), and Decay Speed (gradient of event decay). Signature attributes can be used to determine if a significant event occurred and to help determine the intensity and duration of the event.

As described in the previous flows 100 and 200, video data can be obtained and analyzed for expressions, with methods provided to cluster the expressions together based on various factors such as the type of expression, duration, and intensity. The expression clusters can be plotted. The various plots in the diagram 500 illustrate key information about one or more expression clusters, including a peak value of the expression, the length of the peak value, peak rise and decay, peak rise and decay speed, and so on. Further, based on the clustered expressions, a signature can be determined for the event that occurred while video data was being captured for the plurality of people.

A plot 510 is an example plot of an expression cluster/facial expression probability curve. The facial expression probability curve can be used as a signature. The expression clustering can result from the analysis of video data on a plurality of people based on classifiers, as previously noted. The expression clustering can be for smiles, smirks, brow furrows, squints, lowered eyebrows, raised eyebrows, attention, and so on. The expression clustering can be for a combination of facial expressions. The expression cluster plot 510 can include a time scale 512 and a peak value scale 514, where the time scale can be used to determine a duration, and the peak value scale can be used to determine an intensity for a given expression. The intensity can be based on a numeric scale (e.g. 0-10, or 0-100). In the case of smiles, more exaggerated smile features (for example the amount of lip corner raising that takes place during the smile) can result in a higher intensity value. Analysis of the expression cluster can produce a signature for the event that led to the expression cluster. The signature can include a rise rate, a peak intensity, and a decay rate, for example. Also, the signature can include a time duration. For example, the time duration of the signature determined from the expression plot 510 is the difference in time D between the point 520 and the point 524 on the x-axis of the plot 510. In the plot 510, the point 520 and the point 524 represent adjacent local minima of a facial expression probability curve. Thus, in embodiments, the length of the signature is computed based on detection of adjacent local minima of a facial expression probability curve. The signature can include a peak intensity. For example, the peak intensity of the plot 510 is represented by the point 522, which in this case is a peak value for an expression occurrence. The point 522 can indicate a peak intensity for a smile, a smirk, and so on. In embodiments, a higher peak value for the point 522 indicates a more intense expression in the plot 510, while a lower value for the point 522 indicates a less intense expression value. A difference between a trough intensity value 520 and a peak intensity value 522, as shown in the y-axis peak value scale 514 of the plot 510, can be a component in a signature. The rate of transition from the point 520 to the point 522, and again from the point 522 to the point 524 can be a component of the signature as well, and can help define a shape for an intensity transition from a low intensity to a peak intensity. Additionally, the signature can include a shape for an intensity transition from a peak intensity to a low intensity. The shape of the intensity transition can vary based on the event which is viewed by the people and the type of facial expressions and associated mental states that are occurring. The shape of the intensity transition can vary based on whether the people are experiencing different situations or whether the people are experiencing substantially identical situations. Further, the signature can include a peak intensity and a rise rate to the peak intensity. The rise rate to the peak intensity can indicate a speed for the onset of an expression. Also, the signature can include a peak intensity and a decay rate from the peak intensity, where the decay rate can indicate a speed for the fade of an expression.

Differing clusters are shown in the other plots within FIG. 5. For example, the plot 570 shows an expression that grows significantly in intensity over a long period of time. The plot 570 also shows an end expression value that has a higher intensity than the starting value. Within the cluster 570, the time period to reach an ending value for the expression represents a significant length. Additionally, the peak intensity is shown to be very high and approximately the same for all participants in the data cluster 570, but the beginning values are shown to be widely varying, resulting in a large variance in the expression intensity that can occur in this case. In embodiments, the plot 570 illustrates an instance where a plurality of people with various states of facial activity moved synchronously towards a smile expression and maintained the smile expression for a significant time period. Thus, the signature depicted in the plot 570 can be indicative of an emotional response that gradually builds up over time. Such a response can occur, for example, when listening to a slowly developing humorous story.

Another plot 530 shows a rather uniform change from a trough value to a peak intensity value. The return to a trough value is achieved in roughly the same time as the time to reach a peak intensity. Thus, the signature depicted in the plot 530 can be indicative of an emotional response that quickly occurs and then dissipates. Such a response can occur, for example, when listening to a fairly serious story with a mildly humorous joke unexpectedly interjected.

Still a different plot 540 shows a small change in intensity and a short duration. Some studies indicate that this type of smile is frequently encountered in south-east Asia and the surrounding areas. In this example, the plot 540 can indicate a quick and subtle smile. Yet other plots 550 and 560 show other possible clusters of smiles. The clustering of collective expressions represents a valuable tool by which to understand audience reactions to media and other presentations.

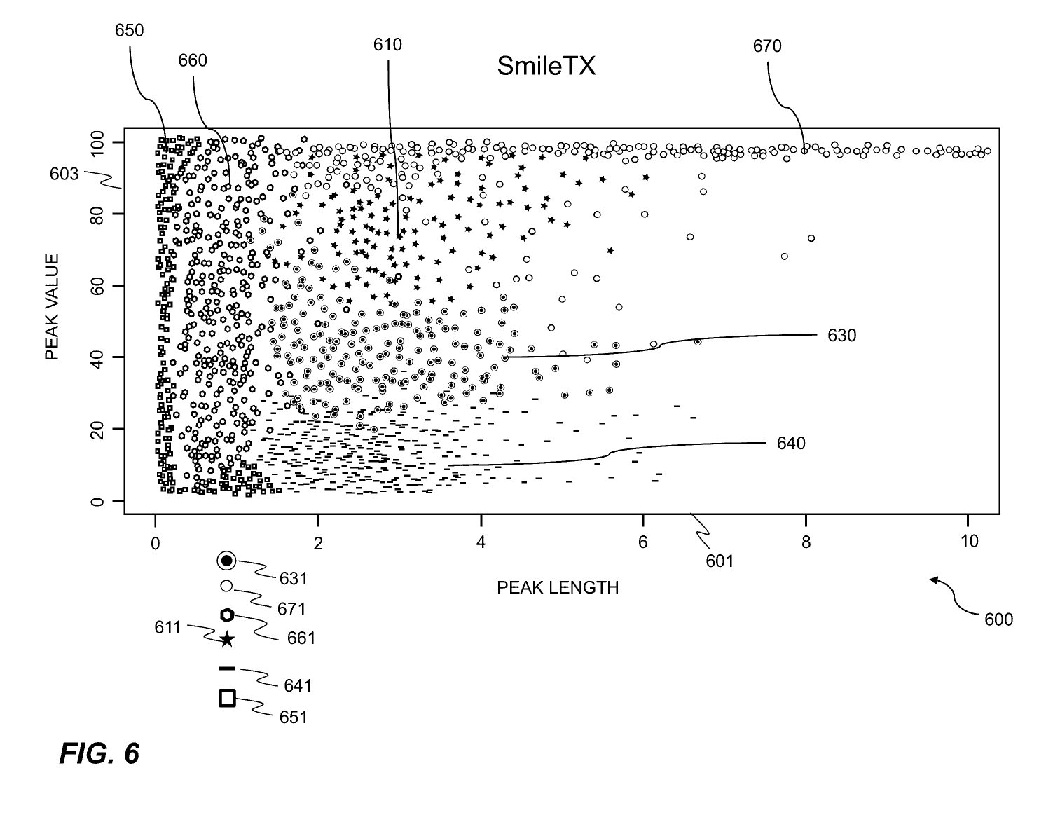

FIG. 6 shows an example plot for smile peak and duration. A plot 600 can be made showing a scatter of expression data resulting from the analyzing of a plurality of videos using classifiers. In the FIG. 600 shown, the plotted expression data includes data for six different events. The event data legend symbols are indicated by the symbols 611, 631, 641, 651, 661, and 671, respectively. Each set of event data corresponds to a plot in FIG. 5. For example, data pertaining to the symbol 611 is associated with the plot 510 of FIG. 5. Data pertaining to the symbol 631 is associated with the plot 530 of FIG. 5. Data pertaining to the symbol 641 is associated with the plot 540 of FIG. 5. Data pertaining to the symbol 651 is associated with the plot 550 of FIG. 5. Data pertaining to the symbol 661 is associated with the plot 560 of FIG. 5. Data pertaining to the symbol 671 is associated with the plot 570 of FIG. 5. The plot 600 shows smile peak duration versus smile peak value. The data point 610 is a representative data point associated with the plot 510 of FIG. 5. The data point 630 is a representative data point associated with the plot 530 of FIG. 5. The data point 640 is a representative data point associated with the plot 540 of FIG. 5. The data point 650 is a representative data point associated with the plot 550 of FIG. 5. The data point 660 is a representative data point associated with the plot 560 of FIG. 5. The data point 670 is a representative data point associated with the plot 570 of FIG. 5. The horizontal axis 601 of the plot 600 represents time in seconds. The vertical axis 603 of the plot 600 represents an intensity value, ranging from a minimum intensity of zero to a maximum intensity of 100. Thus, the plot 600 of FIG. 6 shows a temporal relationship of the intensity of an event signature based on the plotting of expression data.

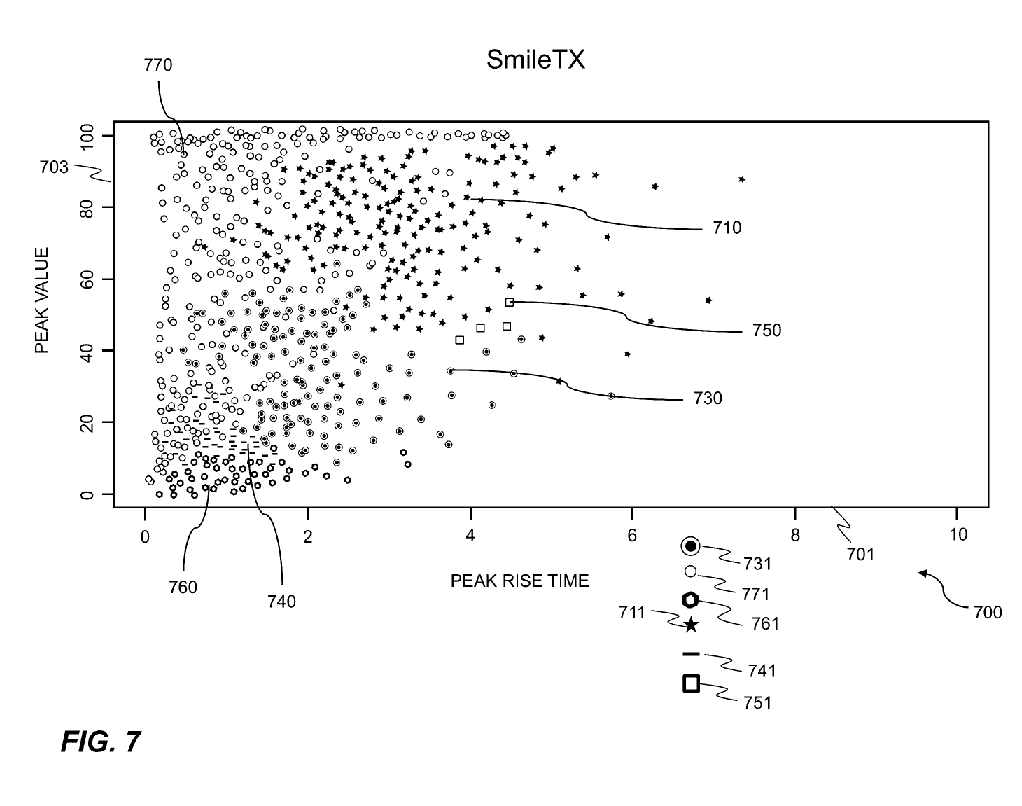

FIG. 7 is an example plot 700 showing peak rise times of smiles. The example 700 illustrates another way of visualizing the data given in FIG. 5. The information shown in the example 700 is a derivative of the temporal relationship of the intensity of an event signature. That is, the example 700 shows the rate of change in expressions over time. As shown here, a plot can be made which illustrates rise speed and peak intensity for an expression. The rise speed will display an onset rate for an expression.

The event data legend symbols are indicated by the symbols 711, 731, 741, 751, 761, and 771. Each set of event data corresponds to a plot in FIG. 5. Data pertaining to the symbol 711 is associated with the plot 510 of FIG. 5. Data pertaining to the symbol 731 is associated with the plot 530 of FIG. 5. Data pertaining to the symbol 741 is associated with the plot 540 of FIG. 5. Data pertaining to the symbol 751 is associated with the plot 550 of FIG. 5. Data pertaining to the symbol 761 is associated with the plot 560 of FIG. 5. Data pertaining to the symbol 771 is associated with the plot 570 of FIG. 5. The plot 700 shows peak rise time versus peak rise for smiles. The data point 710 is a representative data point associated with the plot 510 of FIG. 5. The data point 730 is a representative data point associated with the plot 530 of FIG. 5. The data point 740 is a representative data point associated with the plot 540 of FIG. 5. The data point 750 is a representative data point associated with the plot 550 of FIG. 5. The data point 760 is a representative data point associated with the plot 560 of FIG. 5. The data point 770 is a representative data point associated with the plot 570 of FIG. 5. The horizontal axis 701 of the plot 700 represents time in seconds. The vertical axis 703 of the plot 700 represents an intensity value, ranging from a minimum intensity of zero to a maximum intensity of 100.

In practice, any expression can be plotted for peak rise time versus peak rise, where the expressions can include smiles, smirks, brow furrows, squints, lowered eyebrows, raised eyebrows, attention, and so on. The plot can be used, among other things, to show the effectiveness of an event experienced by a plurality of viewers. In particular, the measure of rise speed can be indicative of a measure of surprise, or a rapid transition of emotional states. For example, in terms of comedic material, a fast peak rise can indicate that a joke was funny, and that it was quickly understood. In the case of dramatic material, a rapid transition to a mental state of surprise or sadness can indicate an unexpected twist in a story.

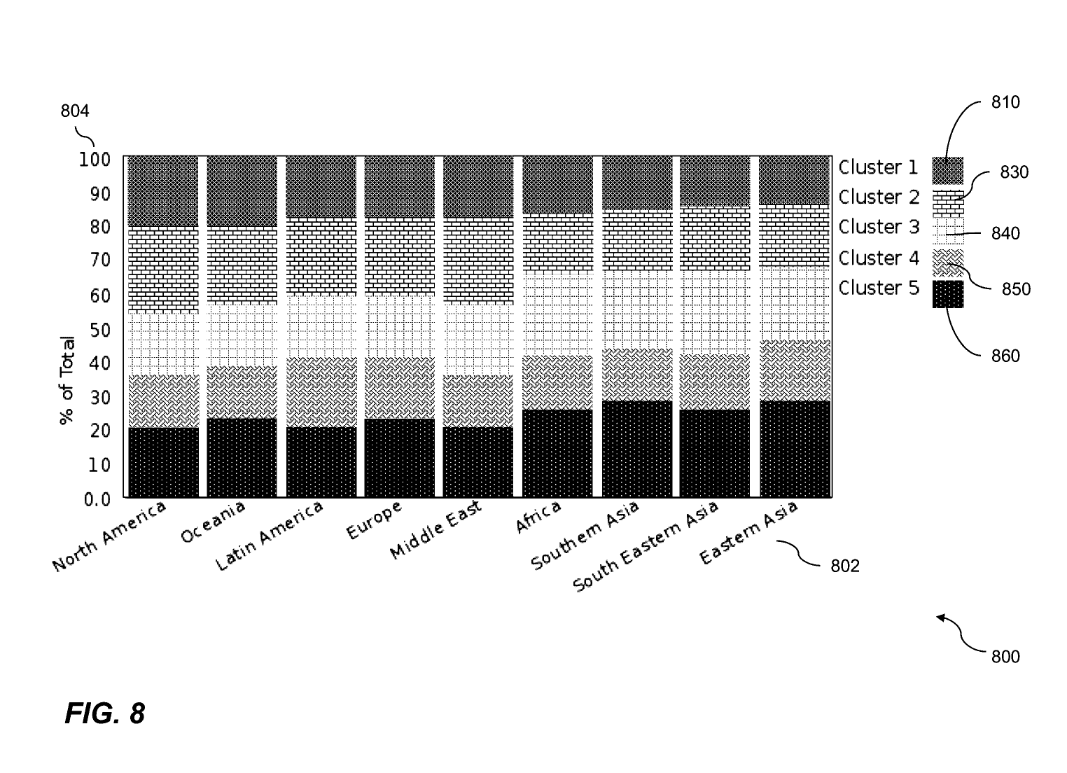

FIG. 8 is an example stacked bar chart 800 showing smile event clusters by region. These smile events are examples of mental state event signature information. In the chart 800, multiple regions are displayed along the horizontal axis 802, and a corresponding percentage for each cluster (810, 830, 840, 850, and 860) is indicated along vertical axis 804. Thus, in the chart 800, the proportion of events from five different clusters that were detected in North America, Oceania, Latin America, Europe, the Middle East, Africa, Southern Asia, Southeastern Asia, and Eastern Asia can be seen. Cultural differences can impact the intensity of emotional expressions. Clustering the different expressions can allow a correlation to be made between an expression and a particular region. For example, some clusters are more prominent in North America than in Asian regions. Conversely, Asian regions can contain a higher ratio of other clusters.

The obtained results suggest that individuals in some regions are generally less expressive than those in other regions. Thus, disclosed embodiments take into account the more subtle examples of given behaviors. As expressiveness is greater in some regions than others, the performance of embodiments that utilize mental state analysis and event signatures can be improved by taking these cultural differences into account.

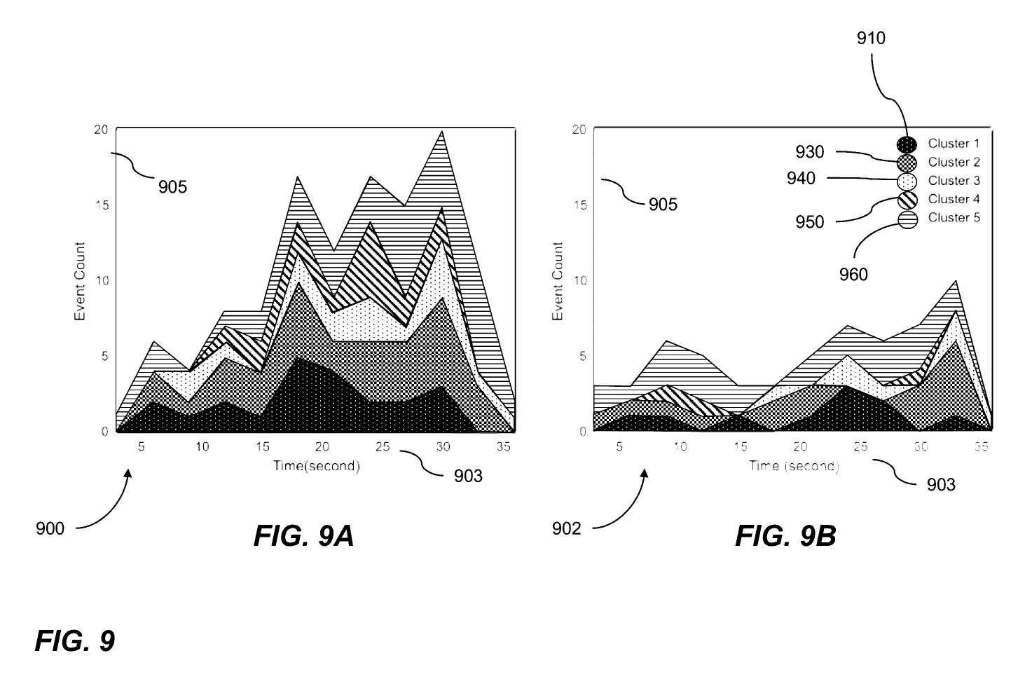

FIG. 9 is an example stacked area chart showing smile event occurrences. FIG. 9 shows stacked area charts displaying the number of occurrences of events from each smile cluster detected during observation of two advertisements. The chart 900 shows the number of occurrences of smiles from five clusters (910, 930, 940, 950, and 960) during viewing of a viral advertisement. A second chart 902 shows the number of occurrences of smiles from the same five clusters (910, 930, 940, 950, and 960) during viewing of a non-viral advertisement. The horizontal axis 903 represents time. The vertical axis 905 represents event counts. Although, in the example shown, both advertisements are intended to be humorous, the non-viral advertisement for which a response is rendered in the chart 902 shows far fewer smile events than the viral advertisement of which the response is rendered in the chart 900. Thus, the identifying the mental state event type can include identification of a weak occurrence of an expression versus a strong occurrence of an expression, as evidenced by both the number and intensity of the expression. For example, the viral advertisement graphed in the chart 900 contains many more events in the clusters 1 910 and 2 930. These two clusters are stronger (more intense) smiles and thus can be classified as more reliable smile events. Thus, in embodiments, the identifying of the mental state event type can be based on a frequency of occurrence of mental state data corresponding to the first event signature (e.g. a smile signature). However, beyond the mere number of smile events, the number of intense smile events can also be used as an indicator of virality. The virality probability index described previously can be further elaborated to account for the number of occurrences of smiles within different clusters. Thus, to account for events in various clusters, the virality probability index can be computed as:

V=K₁(C₁)+K₂(C₂)+K₂(C₃)

where K₁, K₂, and K₃, are constants, and C₁, C₂, and C₃ represent the event counts for different clusters. The constants K₁, K₂, and K₃ can be weighted such that the intense smiles in cluster C₁ are weighted differently than the weaker smiles from cluster C₃. Other factors, such as the shareability factor and prominence index previously described can also be included in the virality probability index along with the individual cluster counts. In such embodiments, the virality probability index can be computed as:

V=K₁(C₁)+K₂(C₂)+K₃(C₃)+K₄(S)+K₅(R)

where S is a shareability factor (how likely the user is to share the video), and R is the prominence index (how famous/relevant the subjects of the video are, and for how long the subjects appear in the video).

Additionally, a weak versus a strong occurrence of an expression can be analyzed on a demographic basis. For example, a weak occurrence of an expression in region A can be determined to be substantially equivalent to a strong occurrence of an expression in region B. Thus, in embodiments, a subtle response is normative for the social group and a more expressive response is provided by an individual where detection of the more expressive response can be used in the identifying the mental state event type. As previously described, different regions can have different normative expression levels. Other demographic groups, such as those based on age and/or gender can also exhibit different tendencies. Embodiments can accommodate these differences in the analysis and usage of mental state data and event signatures.

The human face provides a powerful communications medium through its ability to exhibit a myriad of expressions that can be captured and analyzed for a variety of purposes. In some cases, media producers are acutely interested in evaluating the effectiveness of message delivery by video media. Such video media includes advertisements, political messages, educational materials, television programs, movies, government service announcements, etc. Automated facial analysis can be performed on one or more video frames containing a face in order to detect facial action. Based on the facial action detected, a variety of parameters can be determined including affect valence, spontaneous reactions, facial action units, and so on. The parameters that are determined can be used to infer or predict emotional and mental states. For example, determined valence can be used to describe the emotional reaction of a viewer to a video media presentation or another type of presentation. Positive valence provides evidence that a viewer is experiencing a favorable emotional response to the video media presentation, while negative valence provides evidence that a viewer is experiencing an unfavorable emotional response to the video media presentation. Other facial data analysis can include the determination of discrete emotional states of the viewer or viewers.

Facial data can be collected from a plurality of people using any of a variety of cameras. A camera can include a webcam, a video camera, a still camera, a thermal imager, a CCD device, a phone camera, a three-dimensional camera, a depth camera, a light field camera, multiple webcams used to show different views of a person, or any other type of image capture apparatus that can allow captured data to be used in an electronic system. In some embodiments, the person is permitted to “opt-in” to the facial data collection. For example, the person can agree to the capture of facial data using a personal device such as a mobile device or another electronic device by selecting an opt-in choice. Opting-in can then turn on the person's webcam-enabled device and can begin the capture of the person's facial data via a video feed from the webcam or other camera. The video data that is collected can include one or more persons experiencing an event. The one or more persons can be sharing a personal electronic device or can each be using one or more devices for video capture. The videos that are collected can be collected using a web-based framework. The web-based framework can be used to display the video media presentation or event as well as to collect videos from any number of viewers who are online. That is, the collection of videos can be crowdsourced from those viewers who elected to opt-in to the video data collection.

In some embodiments, a high frame rate camera can be used. A high frame rate camera can be defined as a camera that has a frame rate of 60 frames per second or higher. With such a frame rate, micro expressions can also be captured. Micro expressions are very brief facial expressions, lasting only a fraction of a second. The micro expressions occur when a person either deliberately or unconsciously conceals a feeling.

In some cases, micro expressions happen when people hide their feelings from themselves (repression) or when they deliberately try to conceal their feelings from others. In some cases, the micro expressions might only last about 50 milliseconds. Hence, these expressions often go unnoticed by a human observer. However, a high frame rate camera can be used to capture footage at a sufficient frame rate that the footage can be analyzed for the presence of micro expressions. Micro expressions can be analyzed via action units as previously described, with various attributes such as brow raising, brow furls, eyelid raising, and the like used in the analysis. Thus, embodiments can analyze micro expressions that are easily missed by human observers due to their short durations.

The videos captured from the various viewers who choose to opt-in can be substantially different in terms of video quality, frame rate, etc. As a result, the facial video data can be scaled, rotated, and otherwise adjusted to improve consistency. Human factors further play into the capture of the facial video data. The facial data that is captured might or might not be relevant to the video media presentation being displayed. For example, the viewer might not be paying attention, might be fidgeting, might be distracted by an object or event near the viewer, or otherwise inattentive to the video media presentation. The behavior exhibited by the viewer can prove challenging to analyze due to viewer actions including eating, speaking to another person or persons, speaking on the phone, etc. The videos collected from the viewers might also include other artifacts that pose challenges during the analysis of the video data. The artifacts can include such items as eyeglasses (because of reflections), eye patches, jewelry, and clothing that occlude or obscure the viewer's face. Similarly, a viewer's hair or hair covering can present artifacts by obscuring the viewer's eyes and/or face.

The captured facial data can be analyzed using the facial action coding system (FACS). The FACS seeks to define groups or taxonomies of facial movements of the human face. The FACS encodes movements of individual muscles of the face, where the muscle movements often include slight, instantaneous changes in facial appearance. The FACS encoding is commonly performed by trained observers, but can also be performed on automated, computer-based systems. Analysis of the FACS encoding can be used to determine emotions for the persons whose facial data is captured in the videos. The FACS is used to encode a wide range of facial expressions that are anatomically possible for the human face. The FACS encodings include action units (AUs) and related temporal segments that are based on the captured facial expressions. The AUs are open to higher order interpretation and decision-making. For example, the AUs can be used to recognize emotions experienced by the observed person. Emotion-related facial actions can be identified using the emotional facial action coding system (EMFACS) and the facial action coding system affect interpretation dictionary (FACSAID), for example. For a given emotion, specific action units can be related to the emotion. For example, anger can be related to the expression of AUs 4, 5, 7, and 23, while happiness can be related to the expression of AUs 6 and 12. Other mappings of emotions to AUs have also been previously associated. The coding of the AUs can include an intensity scoring that ranges from A (trace) to E (maximum). The AUs can be used for analyzing images to identify patterns indicative of a particular mental and/or emotional state. The AUs range in number from 0 (neutral face) to 98 (fast up-down look). The AUs include so-called main codes (inner brow raiser, lid tightener, etc.), head movement codes (head turn left, head up, etc.), eye movement codes (eyes turned left, eyes up, etc.), visibility codes (eyes not visible, entire face not visible, etc.), and gross behavior codes (sniff, swallow, etc.). Emotion scoring can be included where intensity is evaluated as well as specific emotions, moods, or mental states.

The coding of faces identified in videos captured of people observing an event can be automated. The automated systems can detect facial AUs or discrete emotional states. The emotional states can include amusement, fear, anger, disgust, surprise, and sadness, for example. The automated systems can be based on a probability estimate from one or more classifiers, where the probabilities can correlate with an intensity of an AU or an expression. The classifiers can be used to identify into which of a set of categories a given observation can be placed. For example, the classifiers can be used to determine a probability that a given AU or expression is present in a given frame of a video. The classifiers can be used as part of a supervised machine learning technique where the machine learning technique can be trained using “known good” data. Once trained, the machine learning technique can proceed to classify new data that is captured.

The supervised machine learning models can be based on support vector machines (SVMs). An SVM can have an associated learning model that is used for data analysis and pattern analysis. For example, an SVM can be used to classify data that can be obtained from collected videos of people experiencing a media presentation. An SVM can be trained using “known good” data that is labeled as belonging to one of two categories (e.g. smile and no-smile). The SVM can build a model that assigns new data into one of the two categories. The SVM can then construct one or more hyperplanes that can be used for classification. The hyperplane that has the largest distance from the nearest training point can be determined to have the best separation. The largest separation can improve the classification technique by increasing the probability that a given data point can be properly classified.

In another example, a histogram of oriented gradients (HoG) can be computed. The HoG can include feature descriptors and can be computed for one or more facial regions of interest. The regions of interest of the face can be located using facial landmark points, where the facial landmark points can include outer edges of nostrils, outer edges of the mouth, outer edges of eyes, etc. A HoG for a given region of interest can count occurrences of gradient orientation within a given section of a frame from a video, for example. The gradients can be intensity gradients and can be used to describe an appearance and a shape of a local object. The HoG descriptors can be determined by dividing an image into small, connected regions, also called cells. A histogram of gradient directions or edge orientations can be computed for pixels in the cell. Histograms can be contrast-normalized based on intensity across a portion of the image or the entire image, thus reducing any influence from illumination or shadowing changes between and among video frames. The HoG can be computed on the image or on an adjusted version of the image, where the adjustment of the image can include scaling, rotation, etc. For example, the image can be adjusted by flipping the image around a vertical line through the middle of a face in the image. The symmetry plane of the image can be determined from the tracker points and landmarks of the image.

In an embodiment, an automated facial analysis system identifies five facial actions or action combinations in order to detect spontaneous facial expressions for media research purposes. Based on the facial expressions that are detected, a determination can be made with regard to the effectiveness of a given video media presentation, for example. The system can detect the presence of the AUs or the combination of AUs in videos collected from a plurality of people. The facial analysis technique can be trained using a web-based framework to crowdsource videos of people as they watch online video content. The video can be streamed at a fixed frame rate to a server. Human labelers can code for the presence or absence of facial actions including symmetric smile, unilateral smile, asymmetric smile, and so on. The trained system can then be used to automatically code the facial data collected from a plurality of viewers experiencing video presentations (e.g. television programs).

Spontaneous asymmetric smiles can be detected in order to understand viewer experiences. Related literature indicates that as many asymmetric smiles occur on the right hemi face as do on the left hemi face, for spontaneous expressions. Detection can be treated as a binary classification problem, where images that contain a right asymmetric expression are used as positive (target class) samples and all other images as negative (non-target class) samples. Classifiers perform the classification, including classifiers such as support vector machines (SVM) and random forests. Random forests can include ensemble-learning methods that use multiple learning algorithms to obtain better predictive performance. Frame-by-frame detection can be performed to recognize the presence of an asymmetric expression in each frame of a video. Facial points can be detected, including the top of the mouth and the two outer eye corners. The face can be extracted, cropped, and warped into a pixel image having a specific dimension (e.g. 96×96 pixels). In embodiments, the inter-ocular distance and vertical scale in the pixel image are fixed. Feature extraction can be performed using computer vision software such as OpenCV™. Feature extraction can be based on the use of HoGs. HoGs can include feature descriptors and can be used to count occurrences of gradient orientation in localized portions or regions of the image. Other techniques can be used for counting occurrences of gradient orientation, including edge orientation histograms, scale-invariant feature transformation descriptors, etc. The AU recognition tasks can also be performed using Local Binary Patterns (LBP) and Local Gabor Binary Patterns (LGBP). The HoG descriptor represents the face as a distribution of intensity gradients and edge directions, and is robust in its ability to translate and scale. Differing patterns, including groupings of cells of various sizes and arrangements of the cells in variously sized cell blocks, can be used. For example, 4×4 cell blocks of 8×8 pixel cells with an overlap of half of the block can be used. Histograms of channels can be used, including nine channels or bins evenly spread over 0-180 degrees. In this example, the HoG descriptor on a 96×96 image is 25 blocks×16 cells×9 bins=3600, the latter quantity representing the dimension. AU occurrences can be rendered. The videos can be grouped into demographic datasets for further detailed analysis based on nationality and/or other demographic parameters.

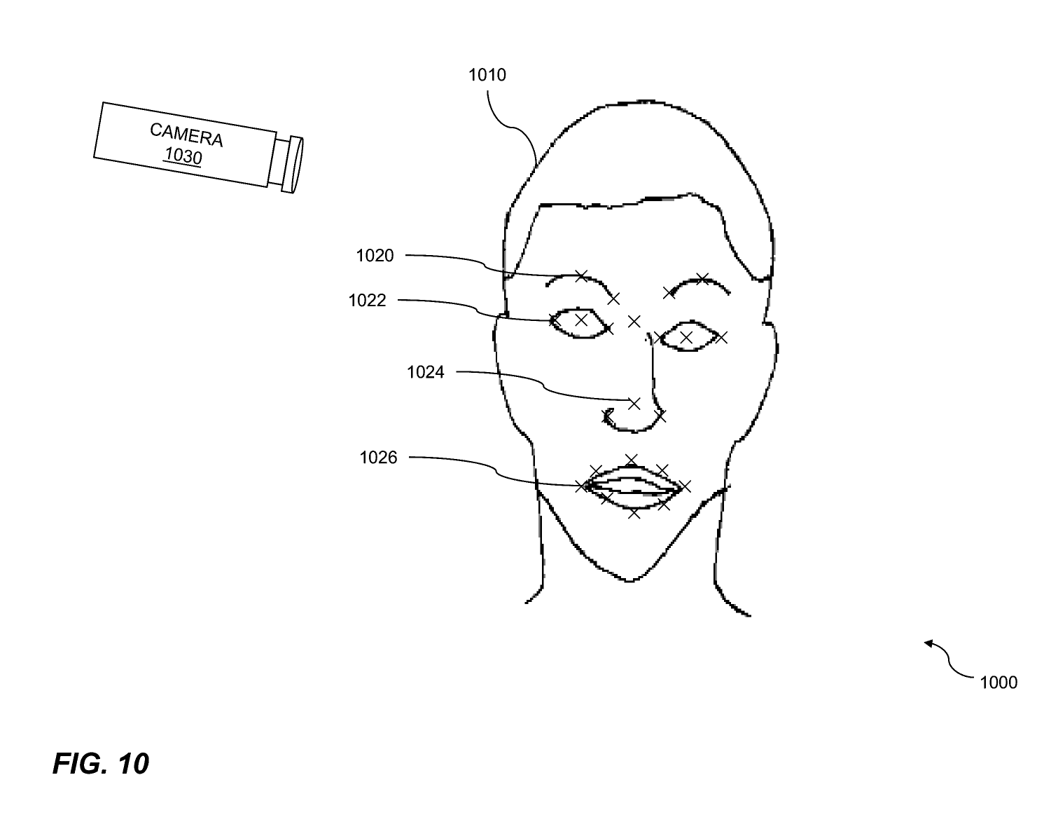

FIG. 10 shows a diagram 1000 illustrating example facial data collection including landmarks. This facial data can be used in mental state event signature analysis. A face 1010 can be observed using a camera 1030 in order to collect facial data that includes facial landmarks. The facial data can be collected from a plurality of people using one or more of a variety of cameras. As discussed above, the camera or cameras can include a webcam, where the webcam can include a video camera, a still camera, a thermal imager, a CCD device, a phone camera, a three-dimensional camera, a depth camera, a light field camera, multiple webcams used to show different views of a person, or any other type of image capture apparatus that can allow captured data to be used in an electronic system. The quality and usefulness of the facial data that is captured can depend, for example, on the position of the camera 1030 relative to the face 1010, the number of cameras used, the illumination of the face, etc. For example, if the face 1010 is poorly lit or over-exposed (e.g. in an area of bright light), the processing of the facial data to identify facial landmarks might be rendered more difficult. In another example, the camera 1030 being positioned to the side of the person might prevent capture of the full face. Other artifacts can degrade the capture of facial data. For example, the person's hair, prosthetic devices (e.g. glasses, an eye patch, and eye coverings), jewelry, and clothing can partially or completely occlude or obscure the person's face. Data relating to various facial landmarks can include a variety of facial features. The facial features can comprise an eyebrow 1020, an outer eye edge 1022, a nose 1024, a corner of a mouth 1026, and so on. Any number of facial landmarks can be identified from the facial data that is captured. The facial landmarks that are identified can be analyzed to identify facial action units. For example, the action units that can be identified include AU02 outer brow raiser, AU14 dimpler, AU17 chin raiser, and so on. Any number of action units can be identified. The action units can be used alone and/or in combination to infer one or more mental states and emotions. A similar process can be applied to gesture analysis (e.g. hand gestures).

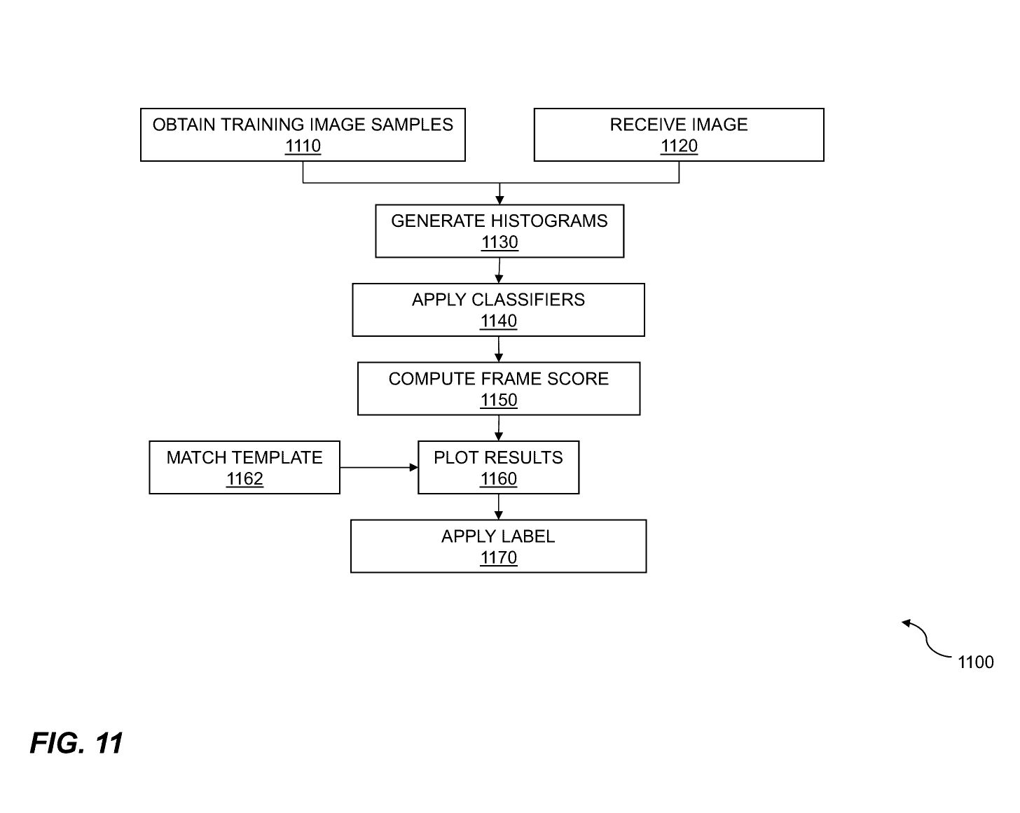

FIG. 11 is a flow diagram describing detection of facial expressions. The flow 1100 illustrates methods and systems for automatically detecting a wide range of facial expressions, which, once detected, can aid in the determining of one or more event signatures. A facial expression can produce strong emotional signals that can indicate valence and discrete emotional states. The discrete emotional states can include contempt, doubt, defiance, happiness, fear, anxiety, and so on. The detection of facial expressions can be based on the location of facial landmarks. The detection of facial expressions can be based on determination of action units (AU) where the action units are determined using FACS coding. The AUs can be used singly or in combination to identify facial expressions. Based on the facial landmarks, one or more AUs can be identified by number and intensity. For example, AU12 can be used to code a lip corner puller and can be used to infer a smirk.

The flow 1100 begins by obtaining training image samples 1110. The training image samples can include a plurality of images of one or more people. Human coders who are trained to correctly identify AU codes based on the FACS can code the images. The training or “known good” images can be used as a basis for training a machine learning technique. Once trained, the machine learning technique can be used to identify AUs in other images that can be collected using a camera, such as the camera 1030 from FIG. 10, for example. The flow 1100 continues with receiving an image 1120. The image 1120 can be received from the camera 1030. As discussed above, the camera or cameras can include a webcam, where a webcam can include a video camera, a still camera, a thermal imager, a CCD device, a phone camera, a three-dimensional camera, a depth camera, a light field camera, multiple webcams used to show different views of a person, or any other type of image capture apparatus that can allow captured data to be used in an electronic system. The image 1120 that is received can be manipulated in order to improve the processing of the image. For example, the image can be cropped, scaled, stretched, rotated, flipped, etc. in order to obtain a resulting image that can be analyzed more efficiently. Multiple versions of the same image can be analyzed. For example, the manipulated image and a flipped or mirrored version of the manipulated image can be analyzed alone and/or in combination to improve analysis. The flow 1100 continues with generating histograms 1130 for the training images and the one or more versions of the received image. The histograms can be generated for one or more versions of the manipulated received image. The histograms can be based on a HoG or another histogram. As described above, the HoG can include feature descriptors and can be computed for one or more regions of interest in the training images and the one or more received images. The regions of interest in the images can be located using facial landmark points, where the facial landmark points can include outer edges of nostrils, outer edges of the mouth, outer edges of eyes, etc. A HoG for a given region of interest can count occurrences of gradient orientation within a given section of a frame from a video, for example.

The flow 1100 continues with applying classifiers 1140 to the histograms. The classifiers can be used to estimate probabilities where the probabilities can correlate with an intensity of an AU or an expression. The choice of classifiers used is based on the training of a supervised learning technique to identify facial expressions, in some embodiments. The classifiers can be used to identify into which of a set of categories a given observation can be placed. For example, the classifiers can be used to determine a probability that a given AU or expression is present in a given image or frame of a video. In various embodiments, the one or more AUs that are present include AU01 inner brow raiser, AU12 lip corner puller, AU38 nostril dilator, and so on. In practice, the presence or absence of any number of AUs can be determined. The flow 1100 continues with computing a frame score 1150. The score computed for an image, where the image can be a frame from a video, can be used to determine the presence of a facial expression in the image or video frame. The score can be based on one or more versions of the image 1120 or manipulated image. For example, the score can be based on a comparison of the manipulated image to a flipped or mirrored version of the manipulated image. The score can be used to predict a likelihood that one or more facial expressions are present in the image. The likelihood can be based on computing a difference between the outputs of a classifier used on the manipulated image and on the flipped or mirrored image, for example. The classifier that is used can be used to identify symmetrical facial expressions (e.g. smile), asymmetrical facial expressions (e.g. outer brow raiser), and so on.

The flow 1100 continues with plotting results 1160. The results that are plotted can include one or more scores for one or frames computed over a given time t. For example, the plotted results can include classifier probability results from an analysis of HoGs for a sequence of images and video frames. The plotted results can be matched with a template 1162. The template can be temporal and can be represented by a centered box function or another function. A best fit with one or more templates can be found by computing a minimum error. Other best-fit techniques can include polynomial curve fitting, geometric curve fitting, and so on. The flow 1100 continues with applying a label 1170. The label can be used to indicate that a particular facial expression has been detected in the one or more images or video frames which constitute the image 1120. For example, the label can be used to indicate that any of a range of facial expressions has been detected, including a smile, an asymmetric smile, a frown, and so on. Various steps in the flow 1100 may be changed in order, repeated, omitted, or the like without departing from the disclosed concepts. Various embodiments of the flow 1100 may be included in a computer program product embodied in a non-transitory computer readable medium that includes code executable by one or more processors.

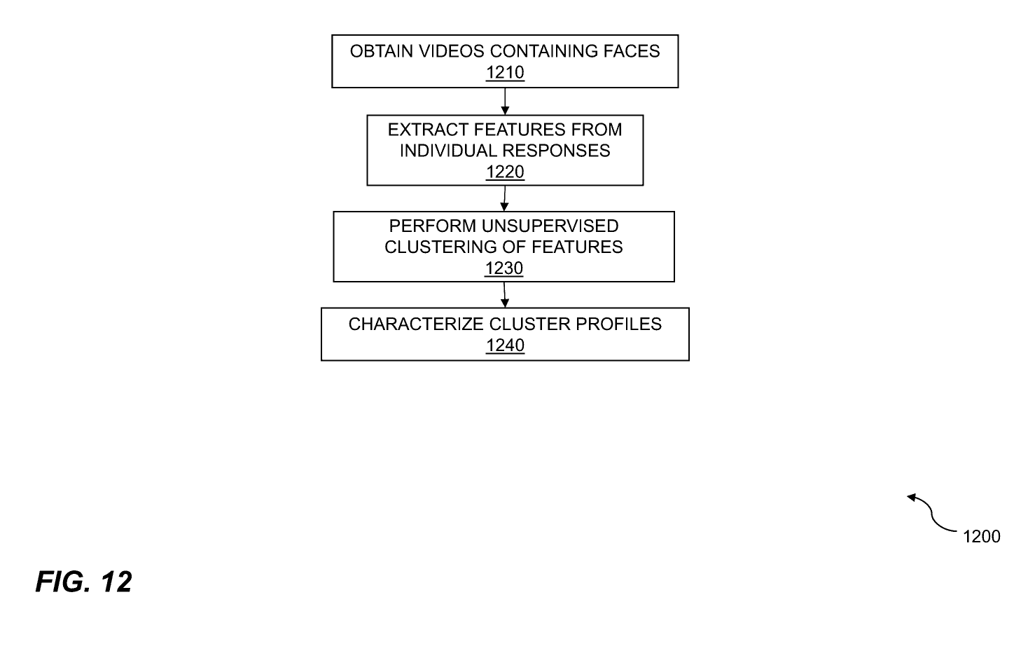

FIG. 12 is a flow 1200 for the large-scale clustering of facial events. As discussed above, collection of facial video data from one or more people can include a web-based framework. The web-based framework can be used to collect facial video data from, for example, large numbers of people located over a wide geographic area. The web-based framework can include an opt-in feature that allows people to agree to facial data collection. The web-based framework can be used to render and display data to one or more people and can collect data from the one or more people. For example, the facial data collection can be based on showing one or more viewers a video media presentation through a website. The web-based framework can be used to display the video media presentation or event and to collect videos from any number of viewers who are online. That is, the collection of videos can be crowdsourced from those viewers who elected to opt-in to the video data collection. The video event can be a commercial, a political ad, an educational segment, and so on. The flow 1200 begins with obtaining videos containing faces 1210. The videos can be obtained using one or more cameras, where the cameras can include a webcam coupled to one or more devices employed by the one or more people using the web-based framework. The flow 1200 continues with extracting features from the individual responses 1220. The individual responses can include videos containing faces observed by the one or more webcams. The features that are extracted can include facial features such as an eyebrow, a nostril, an eye edge, a mouth edge, and so on. The feature extraction can be based on facial coding classifiers, where the facial coding classifiers output a probability that a specified facial action has been detected in a given video frame. The flow 1200 continues with performing unsupervised clustering of features 1230. The unsupervised clustering can be based on an event. The unsupervised clustering can be based on a K-Means, where the K of the K-Means can be computed using a Bayesian Information Criterion (BICk), for example, to determine the smallest value of K that meets system requirements. Any other criterion for K can also be used. The K-Means clustering technique can be used to group one or more events into various respective categories.

The flow 1200 continues with characterizing cluster profiles 1240. The profiles can include a variety of facial expressions such as smiles, asymmetric smiles, eyebrow raisers, eyebrow lowerers, etc. The profiles can be related to a given event. For example, a humorous video can be displayed in the web-based framework and the video data of people who have opted-in can be collected. The characterization of the collected and analyzed video can depend in part on the number of smiles that occurred at various points throughout the humorous video. Similarly, the characterization can be performed on collected and analyzed videos of people viewing a news presentation. The characterized cluster profiles can be further analyzed based on demographic data. For example, the number of smiles resulting from people viewing a humorous video can be compared to various demographic groups, where the groups can be formed based on geographic location, age, ethnicity, gender, and so on. Various steps in the flow 1200 may be changed in order, repeated, omitted, or the like without departing from the disclosed concepts. Various embodiments of the flow 1200 may be included in a computer program product embodied in a non-transitory computer readable medium that includes code executable by one or more processors.

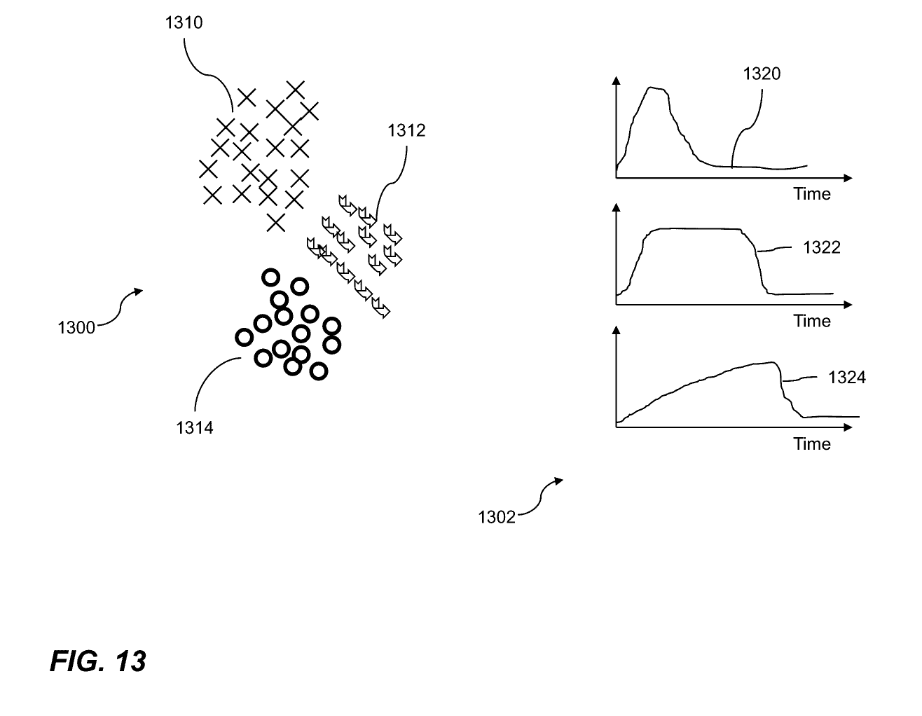

FIG. 13 shows example unsupervised clustering of features and characterization of cluster profiles. Features including samples of facial data can be clustered using unsupervised clustering. Various clusters can be formed, which include similar groupings of facial data observations. The example 1300 shows three clusters 1310, 1312, and 1314. The clusters can be based on video collected from people who have opted-in to video collection. When the data collected is captured using a web-based framework, then the data collection can be performed on a grand scale, including hundreds, thousands, or even more participants who can be located locally and/or across a wide geographic area. Unsupervised clustering is a technique that can be used to process the large amounts of captured facial data and to identify groupings of similar observations. The unsupervised clustering can also be used to characterize the groups of similar observations. The characterizations can include identifying behaviors of the participants. The characterizations can be based on identifying facial expressions and facial action units of the participants. Some behaviors and facial expressions can include faster or slower onsets, faster or slower offsets, longer or shorter durations, etc. The onsets, offsets, and durations can all correlate to time. The data clustering that results from the unsupervised clustering can support data labeling. The labeling can include FACS coding. The clusters can be partially or totally based on a facial expression resulting from participants viewing a video presentation, where the video presentation can be an advertisement, a political message, educational material, a public service announcement, and so on. The clusters can be correlated with demographic information, where the demographic information can include educational level, geographic location, age, gender, income level, and so on.

The cluster profiles 1302 can be generated based on the clusters that can be formed from unsupervised clustering, with time shown on the x-axis and intensity or frequency shown on the y-axis. The cluster profiles can be based on captured facial data including facial expressions, for example. The cluster profile 1320 can be based on the cluster 1310, the cluster profile 1322 can be based on the cluster 1312, and the cluster profile 1324 can be based on the cluster 1314. The cluster profiles 1320, 1322, and 1324 can be based on smiles, smirks, frowns, or any other facial expression. Emotional states of the people who have opted-in to video collection can be inferred by analyzing the clustered facial expression data. The cluster profiles can be plotted with respect to time and can show a rate of onset, a duration, and an offset (rate of decay). Other time-related factors can be included in the cluster profiles. The cluster profiles can be correlated with demographic information as described above.

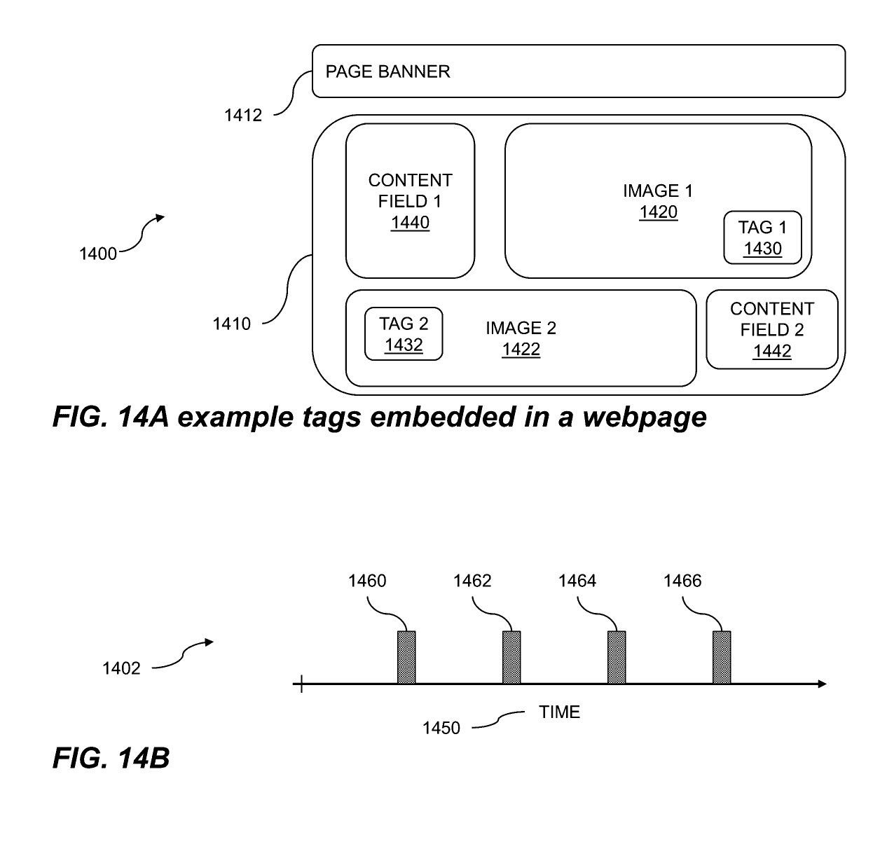

FIG. 14A shows example tags embedded in a webpage where the tags can be used in mental state data collection. A webpage 1400 can include a page body 1410, a page banner 1412, and so on. The page body can include one or more objects, where the objects can include text, images, videos, audio, and so on. The example page body 1410 shown includes a first image, image 1 1420; a second image, image 2 1422; a first content field, content field 1 1440; and a second content field, content field 2 1442. In practice, the page body 1410 can contain any number of images and content fields, and can include one or more videos, one or more audio presentations, and so on. The page body can include embedded tags, such as tag 1 1430 and tag 2 1432. In the example shown, tag 1 1430 is embedded in image 1 1420, and tag 2 1432 is embedded in image 2 1422. In embodiments, any number of tags can be imbedded. Tags can also be imbedded in content fields, in videos, in audio presentations, etc. When a user mouses over a tag or clicks on an object associated with a tag, the tag can be invoked. For example, when the user mouses over tag 1 1430, tag 1 1430 can then be invoked. Invoking tag 1 1430 can include enabling a camera coupled to a user's device and capturing one or more images of the user as the user views a media presentation (or digital experience). In a similar manner, when the user mouses over tag 2 1432, tag 2 1432 can be invoked. Invoking tag 2 1432 can also include enabling a camera and capturing images of the user. In other embodiments, other actions can be taken based on invocation of the one or more tags. For example, invoking an embedded tag can initiate an analysis technique, post to social media, award the user a coupon or another prize, initiate mental state analysis, perform emotion analysis, and so on.

FIG. 14B shows example tag invoking to collect images. As stated above, a media presentation can be a video, a webpage, and so on. A video 1402 can include one or more embedded tags, such as a tag 1460, another tag 1462, a third tag 1464, a fourth tag 1466, and so on. In practice, any number of tags can be included in the media presentation. The one or more tags can be invoked during the media presentation. The collection of the invoked tags can occur over time as represented by a timeline 1450. When a tag is encountered in the media presentation, the tag can be invoked. For example, when the tag 1460 is encountered, invoking the tag can enable a camera coupled to a user device and can capture one or more images of the user viewing the media presentation. Invoking a tag can depend on opt-in by the user. For example, if a user has agreed to participate in a study by indicating an opt-in, then the camera coupled to the user's device can be enabled and one or more images of the user can be captured. If the user has not agreed to participate in the study and has not indicated an opt-in, then invoking the tag 1460 neither enables the camera nor captures images of the user during the media presentation. The user can indicate an opt-in for certain types of participation, where opting-in can be dependent on specific content in the media presentation. For example, the user might opt-in to participation in a study of political campaign messages and not opt-in for a particular advertisement study. In this case, tags that are related to political campaign messages and that enable the camera and image capture when invoked would be embedded in the media presentation. However, tags imbedded in the media presentation that are related to advertisements would not enable the camera when invoked. Various other situations of tag invocation are possible.

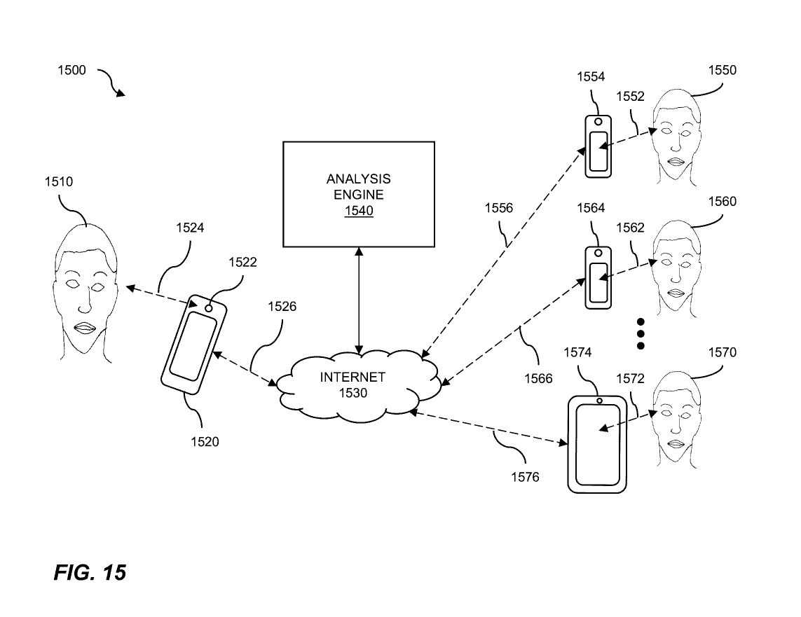

FIG. 15 shows example live-streaming of social video. Such streaming can include mental state event signature analysis. Live-streaming video is an example of one-to-many social media where video can be sent over the Internet from one person to a plurality of people using a social media app and/or platform. Live-streaming is one of numerous popular techniques used by people who want to disseminate ideas, send information, provide entertainment, share experiences, and so on. Some of the live-streams can be scheduled, such as webcasts, online classes, sporting events, news, computer gaming, or videoconferences, while others can be impromptu streams that are broadcast as and when needed or desirable. Examples of impromptu live-stream videos can range from individuals simply wanting to share experiences with their social media followers, to coverage of breaking news, emergencies, or natural disasters. This latter coverage can be known as mobile journalism or “mo jo” and is becoming increasingly commonplace. “Reporters” can use networked, portable electronic devices to provide mobile journalism content to a plurality of social media followers. Such reporters can be quickly and inexpensively deployed as the need or desire arises.

Several live-streaming social media apps and platforms can be used for transmitting video. One such video social media app is Meerkat™ that can link with a user's Twitter™ account. Meerkat™ enables a user to stream video using a handheld, networked, electronic device coupled to video capabilities. Viewers of the live-stream can comment on the stream using tweets that can be seen by and responded to by the broadcaster. Another popular app is Periscope™ that can transmit a live recording from one user to that user's Periscope™ and other followers. The Periscope™ app can be executed on a mobile device. The user's Periscope™ followers can receive an alert whenever that user begins a video transmission. Another live-stream video platform is Twitch™ that can be used for video streaming of video gaming and broadcasts of various competitions and events.

The example 1500 shows user 1510 broadcasting a video live-stream to one or more people 1550, 1560, 1570, and so on. A portable, network-enabled electronic device 1520 can be coupled to a camera 1522 that is forward facing or person facing. The portable electronic device 1520 can be a smartphone, a PDA, a tablet, a laptop computer, and so on. The camera 1522 coupled to the device 1520 can have a line-of-sight view 1524 to the user 1510 and can capture video of the user 1510. The captured video can be sent to an analysis engine 1540 using a network link 1526 to the Internet 1530. The network link can be a wireless link, a wired link, and so on. The analysis engine 1540 can recommend to the user 1510 an app and/or platform that can be supported by the server and can be used to provide a video live-stream to one or more followers of the user 1510. The example 1500 shows three followers 1550, 1560, and 1570 of user 1510. Each follower has a line-of-sight view to a video screen on a portable, networked electronic device. In other embodiments, one or more followers can be following the user 1510 using any other networked electronic device including a computer. In example 1500, person 1550 has line-of-sight view 1552 to the video screen of device 1554, person 1560 has line-of-sight view 1562 to the video screen of device 1564, and user 1570 has line-of-sight view 1572 to the video screen of device 1574. The portable electronic device 1554, 1564, and 1574 each can be a smartphone, a PDA, a tablet, and so on. Each portable device can receive the video stream being broadcast by user 1510 through the Internet 1530 using the app and/or platform that can be recommended by the analysis engine 1540. Device 1554 can receive a video stream using network link 1556, device 1564 can receive a video stream using network link 1566, device 1574 can receive a video stream using network link 1576, and so on. The network link can be a wireless link, and wired link, and so on. Depending on the app and/or platform that can be recommended by the analysis engine 1540, one or more followers, for example, followers 1550, 1560, 1570, and so on, can reply to, comment on, and otherwise provide feedback to the user 1510 using their devices 1554, 1564, and 1574 respectively.

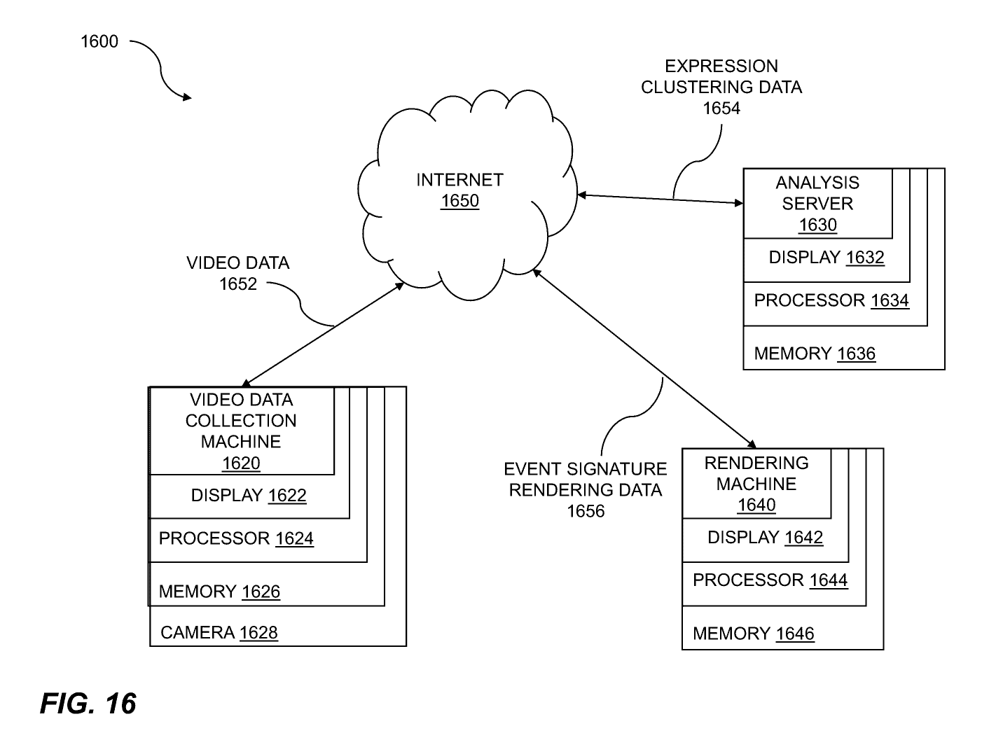

FIG. 16 is a system 1600 for mental state event definition generation. An example system 1600 is shown for mental state event definition collection, analysis, and rendering. The system 1600 can provide a computer-implemented method for analysis comprising: obtaining a plurality of mental state event temporal signatures; collecting mental state data from an individual; comparing the plurality of mental state event temporal signatures against the mental state data; and identifying a mental state event type, based on the plurality of mental state event signatures.

The system 1600 can include a computer system for analysis comprising: a memory which stores instructions; one or more processors attached to the memory wherein the one or more processors, when executing the instructions which are stored, are configured to: obtain a plurality of mental state event temporal signatures; collect mental state data from an individual; compare the plurality of mental state event temporal signatures against the mental state data; and identify a mental state event type, based on the plurality of mental state event signatures.

The system 1600 can include one or more video data collection machines 1620 linked to an analysis server 1630 and a rendering machine 1640 via the Internet 1650 or another computer network. The network can be wired or wireless. Video data 1652 can be transferred to the analysis server 1630 through the Internet 1650, for example. The example video collection machine 1620 shown comprises one or more processors 1624 coupled to a memory 1626 which can store and retrieve instructions, a display 1622, and a camera 1628. The camera 1628 can include a webcam, a video camera, a still camera, a thermal imager, a CCD device, a phone camera, a three-dimensional camera, a depth camera, a light field camera, multiple webcams used to show different views of a person, or any other type of image capture technique that can allow captured data to be used in an electronic system. The memory 1626 can be used for storing instructions, video data on a plurality of people, one or more classifiers, and so on. The display 1622 can be any electronic display, including but not limited to, a computer display, a laptop screen, a net-book screen, a tablet computer screen, a smartphone display, a mobile device display, a remote with a display, a television, a projector, or the like.

The analysis server 1630 can include one or more processors 1634 coupled to a memory 1636 which can store and retrieve instructions, and can also include a display 1632. The analysis server 1630 can receive the video data 1652 and analyze the video data using classifiers. The classifiers can be stored in the analysis server, loaded into the analysis server, provided by a user of the analysis server, and so on. The analysis server 1630 can use video data received from the video data collection machine 1620 to produce expression-clustering data 1654. In some embodiments, the analysis server 1630 receives video data from a plurality of video data collection machines, aggregates the video data, processes the video data or the aggregated video data, and so on.

The rendering machine 1640 can include one or more processors 1644 coupled to a memory 1646 which can store and retrieve instructions and data, and can also include a display 1642. The rendering of event signature rendering data 1656 can occur on the rendering machine 1640 or on a different platform than the rendering machine 1640. In embodiments, the rendering of the event signature rendering data 1656 can occur on the video data collection machine 1620 or on the analysis server 1630. As shown in the system 1600, the rendering machine 1640 can receive event signature rendering data 1656 via the Internet 1650 or another network from the video data collection machine 1620, from the analysis server 1630, or from both. The rendering can include a visual display or any other appropriate display format. The system 1600 can include a computer program product embodied in a non-transitory computer readable medium for analysis comprising code which causes one or more processors to perform operations of: obtaining a plurality of mental state event temporal signatures; collecting mental state data from an individual; comparing the plurality of mental state event temporal signatures against the mental state data; and identifying a mental state event type, based on the plurality of mental state event temporal signatures.

Each of the above methods may be executed on one or more processors on one or more computer systems. Embodiments may include various forms of distributed computing, client/server computing, and cloud based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general purpose hardware and computer instructions, and so on.

A programmable apparatus which executes any of the above mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the forgoing examples should not limit the spirit and scope of the present invention; rather it should be understood in the broadest sense allowable by law.

What is claimed is:

1. A computer-implemented method for analysis comprising:
obtaining a plurality of mental state event temporal signatures;
collecting mental state data from an individual;
comparing the plurality of mental state event temporal signatures against the mental state data; and
identifying a mental state event type, based on the plurality of mental state event temporal signatures.

2. The method of claim 1 further comprising using the mental state event type, which was identified, to perform an evaluation of the individual against other people within a social group.

3. The method of claim 2 wherein the social group is based on demographics, income, job responsibilities, ethnicity, buying behavior, or career objectives.

4. The method of claim 2 further comprising determining a significant difference for the mental state data for the individual versus the social group.

5. The method of claim 2 wherein a first response level is normative for the social group and a second response level is provided by the individual where detection of the second response level is used in the identifying of the mental state event type.

6. The method of claim 5 wherein the first response level is a subtle response relative to the second response level which is a more expressive response.

7. The method of claim 2 further comprising performing an action based on the evaluation of the individual against the other people.

8. (canceled)

9. The method of claim 7 further comprising comparing the individual against a norm for the social group.

10. The method of claim 7 wherein the action is based on a normative score based on the mental state event type.

11. (canceled)

12. The method of claim 7 wherein the action includes computing a virality probability index for a video viewed by the individual while the mental state data is being collected.

13. The method of claim 12 wherein the computing of the virality probability index further comprises computing an emotional response index for the video.

14. The method of claim 13 wherein the computing of the emotional response index comprises:
determining an emotional response curve as a function of time for the video; and
computing an integral of the emotional response curve.

15. The method of claim 13 wherein the computing of the emotional response index comprises:
determining an emotional response curve as a function of time for the video; and
computing a maximum peak level for the emotional response curve.

16-17. (canceled)

18. The method of claim 12 further comprising indicating a video is likely to go viral, in response to computing a virality probability index above a predetermined threshold.

19. The method of claim 12 wherein the computing of the virality probability index further comprises computing a prominence index for people contained in the video.

20. The method of claim 1 further comprising matching a first event signature, from the plurality of mental state event temporal signatures, against the mental state data that was obtained.

21. The method of claim 20 further comprising matching a second event signature, from the plurality of mental state event temporal signatures, against the mental state data that was obtained and identifying the mental state event type based on both the first event signature and second event signature.

22. The method of claim 20 wherein the identifying of the mental state event type is based on a frequency of occurrence of mental state data corresponding to the first event signature.

23. The method of claim 20 wherein the first event signature is based on an image classifier and includes a peak intensity and a duration for an expression.

24. The method of claim 23 wherein the first event signature further includes a rise rate to the peak intensity, a fall rate from the peak intensity, a trough value for intensity, a delta between the trough value for the intensity and the peak intensity, a time delta between the trough value and the peak intensity, a trough value for intensity after peak value, a delta between the peak intensity and the trough value for the intensity after the peak value, a time delta between the peak intensity and the trough value, a time delta between a trough value before the peak intensity and the trough value after the peak value, a beginning of onset and an end of onset timing, a beginning of offset and an end of offset timing, or a sustained period timing.

25. The method of claim 20 wherein the first event signature is used by an SDK.

26. (canceled)

27. The method of claim 20 wherein the first event signature is obtained by performing expression clustering.

28. The method of claim 20 wherein the first event signature is used to detect one or more of sadness, stress, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, curiosity, humor, poignancy, or mirth.

29. The method of claim 20 wherein the identifying the mental state event type includes identification of a weak versus a strong occurrence of an expression.

30. The method of claim 29 wherein the weak versus the strong occurrence of an expression is analyzed on a demographic basis.

31. A computer program product embodied in a non-transitory computer readable medium for analysis comprising code which causes one or more processors to perform operations of:
obtaining a plurality of mental state event temporal signatures;
collecting mental state data from an individual;
comparing the plurality of mental state event temporal signatures against the mental state data; and
identifying a mental state event type, based on the plurality of mental state event temporal signatures.

32. A computer system for analysis comprising:
a memory which stores instructions;
one or more processors attached to the memory wherein the one or more processors, when executing the instructions which are stored, are configured to:
obtain a plurality of mental state event temporal signatures;
collect mental state data from an individual;
compare the plurality of mental state event temporal signatures against the mental state data; and
identify a mental state event type, based on the plurality of mental state event temporal signatures.
